(19) United States
(12) Patent Application Publication
(10) Pub. No.: US 2008/0260842 A1
(43) Pub. Date: Oct. 23, 2008

(54) PERMEATION ENHANCING COMPOSITIONS FOR ANTICHOLINERGIC AGENTS

(76) Inventors: Arnaud Grenier, Steinbrunn-Le-Haut (FR); Dario Norberto R. Carrara, Oberwil (CH); Celine Decaudin, Saint Louis (FR)

Correspondence Address: WINSTON & STRAWN LLP;PATENT DEPARTMENT, 1700 K STREET, N.W., WASHINGTON, DC, 20006, US

(21) Appl. No.: 12/147,816

(22) Filed: Jun. 27, 2008

Related U.S. Application Data
(63) Continuation-in-part of application No. 11/120,306, filed on May 2, 2005.
(60) Provisional application No. 60/568,983, filed on May 7, 2004.

(51) Int. Cl. A61K 9/50; A61K 47/16; A61K 47/12; A61P 13/10; A61K 31/24; A61K 9/12

(52) U.S. Cl. 424/490; 514/788; 514/784; 514/534; 514/648; 424/45

(57) ABSTRACT

A transdermal or topical skin-friendly composition including anticholinergic agents, such as oxybutynin, a urea-containing compound and a carrier system. A method is disclosed for treating a subject for urinary incontinence while reducing the incidences of peak concentrations of drug and undesirable side effects associated with oral anticholinergics.

### CROSS-REFERENCE TO RELATED APPLICATIONS

This application is a continuation-in-part of U.S. patent application Ser. No. 11/120,306, filed May 2, 2005, which claims the benefit of U.S. Provisional Patent Application Ser. No. 60/568,983, filed May 7, 2004, the contents of each of which are incorporated herein by reference thereto.

### TECHNICAL FIELD

This invention relates generally to compositions comprising anticholinergic or antispasmodic agents, and more particularly relates to compositions that enhance the permeability of skin or mucosal tissue to topically or transdermally applied anticholinergic or antispasmodic agents, and in particular oxybutynin. The invention also relates to methods for treating overactive bladder and urinary incontinence by the administration of the topical or transdermal composition.

### BACKGROUND OF THE INVENTION

Topical or transdermal delivery systems for the administration of drugs are known to offer several advantages over oral delivery of the same drugs. Generally, the advantages of topical or transdermal delivery of drugs relate to pharmacokinetics. More specifically, one common problem associated with the oral delivery of drugs is the occurrence of peaks in serum levels of the drug, which is followed by a drop in serum levels of the drug due to its elimination and possible metabolism. Thus, the serum level concentrations of orally administered drugs have peaks and valleys after ingestion. These highs and lows in serum level concentrations of drug often lead to undesirable side effects.

In contrast, topical and transdermal delivery of drugs provides a relatively slow and steady delivery of the drug. Accordingly, unlike orally administered drugs, the serum concentrations of topically or transdermally delivered drugs are substantially sustained and do not have the peaks associated with oral delivery.

The sustained serum concentrations associated with topical or transdermal drug delivery avoids the systemic side effects of oral administration of drugs. Specifically, first pass metabolism of the drug by the liver is circumvented by utilizing transdermal or topical delivery vehicles for the administration of drugs.

For example, there is a significant decrease in adverse effects associated with the transdermal delivery of oxybutynin. Oral oxybutynin has been indicated for the relief of symptoms of bladder instability associated with voiding in patients with uninhibited neurogenic or reflex neurogenic bladder, i.e., urgency, frequency, urinary leakage, urge incontinence, and dysuria.

It has been shown that transdermal administration of oxybutynin results in a substantially lower fluctuation in the plasma concentrations of oxybutynin and its metabolite N-desethyloxybutynin than oral delivery of oxybutynin. Additionally, reduced N-desethyloxybutynin formation, greater saliva production during the dosing period and lower incidences of dry mouth in patients with overactive bladder were also reported, as compared with oral oxybutynin administration.

Oxybutynin has been found to have a direct antispasmodic effect on smooth muscle and inhibits the muscarinic action of acetylcholine on smooth muscle, but exhibits only one-fifth of the anticholinergic activity of atropine detrusor muscle (effect observed in rabbits), and four to ten times its antispasmodic activity. Oxybutynin has not been found to possess blocking effects at skeletal neuromuscular junctions or autonomic ganglia (antinicotinic effects). Moreover, oxybutynin has been found to relax bladder smooth muscle. In patients with conditions characterized by involuntary bladder contractions, cystometric studies have demonstrated that oxybutynin increases bladder (vesical) capacity, diminishes the frequency of uninhibited contractions of the detrusor muscle, and delays the initial desire to void. Oxybutynin thus decreases urgency and the frequency of both incontinent episodes and voluntary urination. It has also been reported that antimuscarinic activity resides predominantly in the R-isomer.

Adverse reactions associated with oxybutynin therapy, however, may include cardiovascular manifestations such as palpitations, tachycardia or vasodilatation; dermatologic manifestations such as decreased sweating, rash; gastrointestinal/genitourinary manifestations such as constipation, decreased gastrointestinal motility, dry mouth, nausea, urinary hesitance and retention; nervous system manifestations such as asthenia, dizziness, drowsiness, hallucinations, insomnia, restlessness; opthalmic manifestations such as amblyopia, cycloplegia, decreased lacrimation, mydriasis. Most common side effects associated with oral oxybutynin encompasses dry mouth, dizziness, blurred vision, and constipation. These adverse experiences may be uncomfortable enough to persuade the patient to discontinue treatment.

In a study which compared transdermal delivery and oral delivery of oxybutynin, a substantially lower fluctuation in oxybutynin and its metabolite N-desethyloxybutynin plasma concentrations was demonstrated with the transdermally administered oxybutynin. Additionally, reduced N-desethyloxybutynin formation, and greater saliva production during the dosing period was reported compared with oral oxybutynin administration. Moreover, lower incidences of dry mouth in patients with overactive bladder were reported. See, Appel R A, Chancellor M B, Zobrist R H, Thomas H, Sanders S W, “Pharmacokinetics, Metabolism, and Saliva Output during Transdermal and Extended-Release Oral Oxybutynin Administration in Healthy Subjects”, Mayo Clin. Proc. 2003;78: 696-702.

Moreover, Dmochowsky et al. confirmed the improvement of overactive bladder symptoms and quality of life (dry mouth incidence reduction) in patients treated with transdermal oxybutynin compared to oral oxybutynin therapy. See, Dmochowski R R, Davila G W, Zinner N R, Gittelman M C, Saltzstein D R, Lyttle S, Sanders S W; For The Transdermal Oxybutynin Study Group; “Efficacy and safety of transdermal oxybutynin in patients with urge and mixed urinary incontinence”, The Journal of Urology, Vol. 168, 580-586, August 2002. Thus, it can be easily seen that transdermal delivery of oxybutynin has been shown to be more advantageous, as well as preferred over oral delivery of oxybutynin.

Various permeation enhancers have been reported for transdermal or topical delivery of oxybutynin. For example, U.S. Pat. Nos. 5,411,740, 5,500,222, and 5,614,211, each disclose monoglyceride or a mixture of monoglycerides of fatty acids as the preferred permeation enhancer for an oxybutynin transdermal therapeutic system. U.S. Pat. No. 5,736,577 describes a pharmaceutical unit dosage form for transdermal administration of (S)-oxybutynin comprising a permeation enhancer. U.S. Pat. Nos. 5,834,010 and 6,555,129 both disclose triacetin as a permeation enhancer for oxybutynin. U.S. Pat. No. 5,747,065 discloses monoglycerides and lactate esters as a permeation enhancing mixture for oxybutynin.

Moreover, U.S. Pat. No. 5,843,468 describe a dual permeation enhancer mixture of lauryl acetate and a glycerol monolaurate for transdermal administration of oxybutynin. U.S. Pat. No. 6,004,578 disclose permeation enhancers selected from the group consisting of alkyl or aryl carboxylic acid esters of polyethyleneglycol monoalkyl ether, and polyethyleneglycol alkyl carboxymethyl ethers for a transdermal matrix drug delivery device comprising oxybutynin. Meanwhile, U.S. Pat. No. 6,267,984 discloses skin permeation enhancer compositions comprising a monoglyceride and ethyl palmitate for transdermal delivery of oxybutynin. U.S. Pat. No. 6,562,368 discloses the use of hydroxide-releasing agent to increase the permeability of skin or mucosal tissue to transdermally administered oxybutynin. As mentioned above, currently, the approach to finding a suitable permeation enhancer for a particular drug is through trial and error.

Urea is a natural substance and a final metabolite of proteins in the body. The value of urea in pharmaceutical and cosmetic preparations has been recognized since the early days of folk medicine, e.g., urea aids in debridement, dissolves the coagulum and promotes epithelialization when used in a concentration of approximately 10-15 percent; at higher concentrations, e.g., above 40 percent, urea is proteolytic and therefore, is commonly used for the treatment of nail destruction and dissolution, urea is also used as an osmotic diuretic.

One remarkable property of urea is the increased water-holding capacity of the stratum corneum in the presence of urea. Urea is mildly keratolytic and increases water uptake in the stratum corneum. This gives the stratum corneum a high water-binding capacity. Accordingly, urea is often used as a skin moisturizer.

Urea is also generally known as a permeation enhancer for certain drugs. However, the percutaneous absorption enhancement by urea is strongly dependent on the cosolvents used. For example, Kim et al. observed that the penetration of ketoprofen was enhanced in the presence of urea in aqueous solutions, whereas in propylene glycol or propylene glycol-ethanol mixtures no enhancement was reported. Moreover, Kim found that the addition of high amounts of urea increases the diffusivity of ketoprofen.

U.S. Pat. Nos. 7,029,694 and 7,179,483 each to Ebert et al. relate to oxybutynin gel formulations that provide and plasma area under the curve (AUC) ratio of oxybutynin to an oxybutynin metabolite of from about 0.5:1 to about 5:1. These gel formulations include permeation enhancers as optional components. Although Ebert does disclose in general terms various permeation enhancers, solvents and other formulation additives for a carrier such as a water/ethanol gel, which additives may include diethylene glycol monoethyl ether, propylene glycol or urea, Ebert does not provide any disclosure or examples of the necessity of using these components together in combination. Ebert exemplifies triacetin and monoglycerides as preferred permeation enhancers.

A further description of permeation enhancers and their performance can be found in the Background of application Ser. No. 11/120,306 and is incorporated herein by reference.

As known in the art, the transdermal administration of drugs has certain drawbacks associated with drug penetration across the dermal barrier. Skin is a structurally complex multilayered organ with a total thickness of 2-3 mm. Thus, there is continued interest in the development of strategies to enhance the permeation of compounds across the skin barrier.

Although a large numbers of permeation enhancers are generally known in the art, the selection of the most efficient permeation enhancer for a particular drug still relies on empirical techniques through “trial and error” because the efficiencies of known permeation enhancers are unpredictable, and vary with parameters such as (1) the drug to be administered; (2) the concentration of the permeation enhancer; (3) the vehicle or carrier and (4) other components of the delivery system.

Thus, there is a need in the industry for improved topical or transdermal compositions of anticholinergic agents, such as oxybutynin, with enhanced permeation efficiency and sustained transdermal drug flux.

### SUMMARY OF INVENTION

The present invention provides a non-occlusive composition for topical or transdermal administration of an anticholinergic or antispasmodic agent, the composition comprising (1) at least one anticholinergic or antispasmodic agent; (2) a urea-containing compound; and (3) a carrier suitable for transdermal or topical administration. The carrier comprises the combination of an alcohol, a polyalcohol, and either a monoalkyl ether of diethylene glycol or a tetraglycol furol present in the carrier in a combined amount effective with the amount of urea-containing compound to enhance permeation of the anticholinergic or antispasmodic agent through dermal or mucosal surfaces.

In one embodiment of the present invention, the anticholinergic or antispasmodic agent is at least one of oxybutynin, flavoxate, imipramine, propantheline, phenylpropanolamine, darifenacin, duloxetine, tolterodine tartrate, trospium, or solifenacin succinate or a pharmaceutically acceptable salt thereof.

The urea-containing compound is preferably urea, 1,3-dimethylurea, 1,1-diethylurea, 1-acetyl-1-phenylurea, isopropylideneurea, allophanic acid, hydantoic acid, allophanoyl, pyrrolidone carboxylic acid, biuret, thiobiuret, dithiobiuret, triuret and 2-(3-methylureido)-1-naphthoic acid. The urea-containing compound is preferably present in an amount of between about 1 to 20% by weight of the composition.

The carrier comprises the combination of an alcohol, a polyalcohol, and either a monoalkyl ether of diethylene glycol or a tetraglycol furol present in the carrier in a combined amount effective with the amount of urea-containing compound to enhance permeation of the anticholinergic or antispasmodic agent through dermal or mucosal surfaces.

The present invention further provides a method for treating overactive bladder or urge and urinary incontinence in a subject, the method comprising administering to a subject in need thereof, a topical or transdermal composition of oxybutynin according to the present invention.

Advantageously, the composition of the present invention provides a steady plasma concentration of oxybutynin to a subject administered with the composition, as well as avoiding undesirable peaks in drug concentration, and/or reduces the incidences of unwanted, undesirable side effects such as dry mouth, accommodation disturbances, nausea and dizziness.

The oxybutynin composition of the present invention provides a sustained transdermal oxybutynin flux allowing therapeutic levels of oxybutynin for at least 24 hours, preferably for at least 48 hours and most preferably for at least 72 hours. Thus, the composition only needs to be administrated once per day, once every other day, once every third day or twice per week.

### BRIEF DESCRIPTION OF THE DRAWINGS

FIG. 1 is a graph illustrating the results from an in-vitro 24-hour comparative permeation study comparing permeation of a composition comprising oxybutynin, urea, and a carrier, and a composition comprising oxybutynin, a carrier, and no urea;

FIG. 2 is a graph illustrating the results of an in-vitro 24-hour comparative permeation study of a composition comprising oxybutynin, a hydroalcoholic carrier, and solvents, and a composition comprising oxybutynin, urea, a hydroalcoholic carrier and solvents;

FIG. 3 is a graph illustrating the drug flux profiles of the compositions of FIG. 2;

FIG. 4 is a graph illustrating the results of an in-vitro 24-hour comparative permeation study comparing permeation of a composition comprising oxybutynin, and a carrier, a composition comprising oxybutynin, urea and a carrier; and a composition comprising oxybutynin, urea, a carrier and additional solvents;

FIG. 5 is a graph illustrating the drug flux profiles of the compositions of FIG. 4;

FIG. 6 is a graph illustrating the results of a comparative study comparing the absolute kinetic profile of a formulation comprising oxybutynin and urea, a formulation including oxybutynin and lauric acid, and a formulation including oxybutynin and isopropyl myristate;

FIG. 7 is a graph illustrating the flux profile of the formulations of FIG. 6;

FIG. 8 is a graph illustrating the results of a comparative study comparing the absolute kinetic profile of a formulation including oxybutynin and urea, a formulation including oxybutynin and triacetin and a formulation including oxybutynin and glycerol monooleate;

FIG. 9 is a graph illustrating the flux profile of the formulations of FIG. 8;

FIG. 10 is a graph illustrating the relative kinetic profile of a tolterodine formulation including urea as a permeation enhancer;

FIG. 11 is a graph illustrating the relative kinetic profile of tolterodine formulations including a urea derivative;

FIG. 12 is a graph illustrating the drug flux profile of the formulations of FIG. 11;

FIG. 13 is a graph illustrating the results of a pharmacokinetic study of mean plasmatic N-desethyloxybutynin concentrations in ng/ml over a 7-day period;

FIG. 14 is a graph illustrating the results from a pharmacokinetic study of the mean plasmatic Oxybutynin/N-desethyloxybutynin ratio over a 7-day period;

FIG. 15 is a graph illustrating the results of a pharmacokinetic study of mean plasmatic Oxybutynin/N-desethyloxybutynin ratio over a 7-day period;

FIG. 16 shows OXY and DEO plasma concentrations during transdermal gel administration of 42 mg oxybutynin once a day. Values represent the mean (±SD) from 16 subjects;

FIG. 17 is a graph illustrating the results of a pharmacokinetic study of the mean plasmatic oxybutynin concentrations in ng/ml over a 7-day period;

FIG. 18 shows OXY and DEO plasma concentrations during transdermal gel administration of 60 mg oxybutynin once a day. Values represent the mean (±SD) from 16 subjects;

FIG. 19 shows OXY and DEO plasma concentrations during transdermal gel administration of 84 mg oxybutynin once a day. Values represent the mean (±SD) from 16 subjects;

FIG. 20 shows oxybutynin mean plasma concentrations following application of 2.8 g oxybutynin 3% transdermal gel formulation of the present invention over 700 cm² of different skin sites. Values represent the mean (±SD) from 25 subjects;

FIG. 21 shows OXY and DEO mean plasma concentrations following application of 2.8 g oxybutynin 3% transdermal gel formulation of the present invention over 700 cm² of abdomen. Values represent the mean (±SD) from 10 subjects; and

FIG. 22 is an indirect comparison of peak plasma levels (Cₘₐₓ) of R- and S-enantiomers of OXY and DEO after single-dose of either oral (DITROPAN IR tablets 5 mg) or transdermal (2.8 g oxybutynin 3% transdermal gel formulation of the present invention) administration.

### DETAILED DESCRIPTION OF THE PREFERRED EMBODIMENTS

A transdermal or topical composition comprising anticholinergic or antispasmodic agents, such as oxybutynin, is very desirable. As transdermal and topical compositions bypass the gastrointestinal tract, and are not subject to the “first pass hepatic effect,” the blood concentration peaks of the anticholinergic or antispasmodic agent avoided. It has been found that the blood concentration peaks of substances such as oxybutynin often lead to the occurrence of undesirable side-effects, such as dry mouth, accommodation disturbances, nausea and dizziness. Accordingly, the one advantage of bypassing the first-pass metabolism in the liver is the increased bioavailability of drug in comparison to oral administration of drug. As the bioavailability is increased for transdermal or topical administered drugs, the total daily dosages that are necessary for reaching a desired therapeutic effect is reduced. Moreover, since the present compositions comprising anticholinergic or antispasmodic agents, a urea-containing compound and a suitable carrier, provide enhanced permeability and thus even more bioavailability of the drug, the advantages for the transdermal or topical composition of the present invention are even greater.

To achieve improved skin permeation and steady plasma concentration of anticholinergic or antispasmodic agents, the present invention provides for a composition for topical or transdermal administration of an anticholinergic or antispasmodic agent comprising at least one anticholinergic or antispasmodic agent and a permeation enhancing system.

The anticholinergic or antispasmodic agent can be oxybutynin, flavoxate, imipramine, propantheline, phenylpropanolamine, darifenacin, duloxetine, tolterodine tartrate, trospium, or solifenacin succinate or a pharmaceutically acceptable salt thereof, or other agents such as nitric oxide derivatives of flurbiprofen (a prostaglandin synthesis inhibitor) and imipramine (an antidepressant with marked systemic antimuscarinic actions), or a pharmaceutically acceptable salt thereof. Examples of some pharmaceutically acceptable salts are acetate, bitartrate, citrate, edetate, edisylate, estolate, esylate, fumarate, gluceptate, gluconate, glutamate, hydrobromide, hydrochloride, lactate, malate, maleate, mandelate, mesylate, methylnitrate, mucate, napsylate, nitrate, pamoate, pantothenate, phosphate, salicylate, stearate, succinate, sulfate, tannate and tartrate. The skin permeation of these related agents are similarly enhanced by the permeation enhancing system of the invention.

Advantageously, these anticholinergic or antispasmodic agents have been indicated for conditions including overactive bladder and urinary incontinence to mention a couple (Table 1). Thus, the compositions of the invention, which comprises a permeation enhancing system, is not only novel but is desirable since the compositions can now be formulated containing lower amounts of drug.

Preferably, the anticholinergic or antispasmodic agent is oxybutynin, oxybutynin free base or a pharmaceutically acceptable salt thereof. The oxybutynin may be as a racemate or a single isomer such as the S-enantiomer or R-enantiomer.

TABLE 1
Some Active Drugs for Treatment
of Over Active Bladder and Urge Incontinence

| | |
|---|---|
| Oxybutynin | 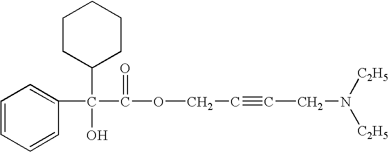 |
| Flavoxate | 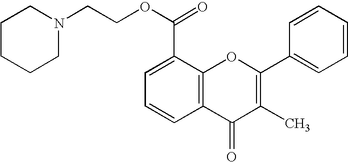 |
| Imipramine | 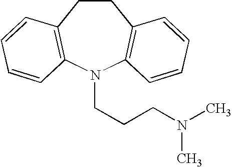 |
| Propantheline | 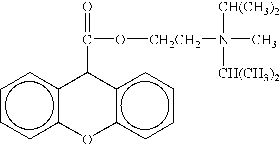 |
| Phenylpropanolamine | 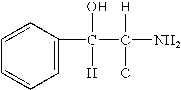 |
| Darifenacin | 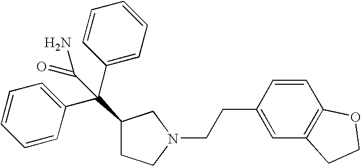 |
| Duloxetine | 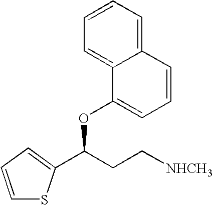 |
| Tolterodine tartrate | 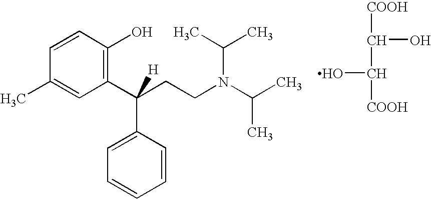 |
| Trospium chloride | 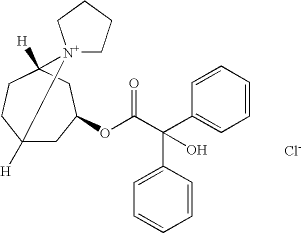 |
| Solifenacin succinate | 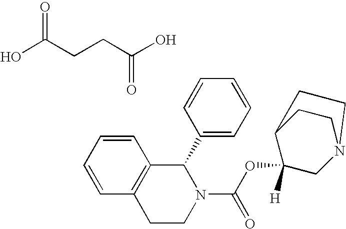 |

The permeation enhancing system of the invention comprises a urea-containing compound and a carrier comprising a combination of an alcohol, a polyalcohol, and either a monoalkyl ether of diethylene glycol or a tetraglycol furol present in an effective amount to enhance permeation of the anticholinergic or antispasmodic agent through dermal or mucosal surfaces, and water.

The urea containing compound has the general formula:

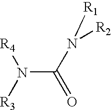

wherein R₁, R₂, R₃, and R₄ is a functional group selected from the group including hydrogen, an alkyl group, a thiol group, an aromatic group, a carboxyl group, a carbonyl group, an ether linkage, an ester group, an amine group, an allophanamide, a glycolyl group, a carbonic acid, or any combination thereof.

For the purpose of illustration and not limitation the urea-containing compound can be urea, a derivative, or an analogue, thereof including 1,3-Dimethylurea, 1,1-Diethylurea, 1-Acetyl-1-phenylurea, Isopropylideneurea, Allophanic acid, Hydantoic acid, Allophanoyl, Pyrrolidone carboxylic acid, Biuret, Thiobiuret, Dithiobiuret, Triuret and 2-(3-Methylureido)-1-naphthoic acid or a derivative thereof as illustrated in Table 2 below.

TABLE 2
Urea Derivatives and Analogues

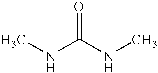

1,3-Dimethylurea

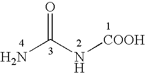

Allophanic Acid

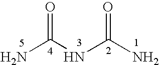

Biuret

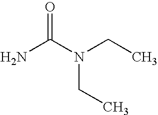

1,1 Diethylurea

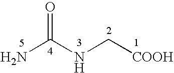

Hydantoic acid

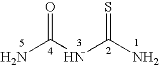

Thiobiuret

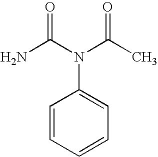

1-Acetyl-1-phenylurea

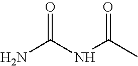

Allophanoyl

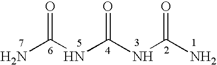

Dithiobiuret

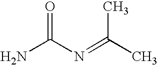

Isopropylideneurea

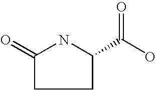

Pyrrolidone carboxylic acid

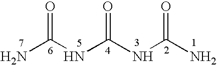

Triuret

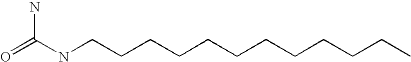

Lauryl urea

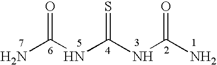

4-Thiotriuret

In accordance with the invention, the carrier is suitable for transdermal or topical administration or delivery of the anticholinergic or antispasmodic agent. The carrier comprises at least one of an alcohol, a polyalcohol, a monoalkyl ether of diethylene glycol, a tetraglycol furol, or water. The phrase “monoalkylether of diethylene glycol” refers to a substance having a general formula C₄H₁₀O₃(CₙH₂ₙ₊₁), wherein n is 1-4. The term “tetraglycol” refers to glycofurol, or tetrahydrofurfuryl alcohol. Further, the term “glycol” encompasses a broad range of chemicals including but not limited to propylene glycol, dipropylene glycol, butylene glycol, and polyethylene glycols having general formula CH₂OH(CH₂OH)ₙCH₂OH wherein the number of oxyethylene groups represented by n is between 4 to 200.

Preferably, the alcohol is ethanol, propanol, isopropanol, 1-butanol, 2-butanol or a mixture thereof, and more preferably is ethanol. Preferably, the polyalcohol is propylene glycol, dipropylene glycol or a mixture thereof, and more preferably is propylene glycol. Preferably, the monoalkyl ether of diethylene glycol is diethylene glycol monomethyl ether, diethylene glycol monoethyl ether or a mixture thereof, and more preferably is diethylene glycol monoethyl ether. Preferably, the tetraglycol furol is glycofurol. Water can be added to constitute the balance of the carrier.

Preferably, the carrier of the invention comprises the combination of an alcohol, a polyalcohol, a monoalkyl ether of diethylene glycol or a tetraglycol furol, and water. Alternatively, the carrier of the invention comprises the combination of a polyalcohol, a monoalkyl ether of diethylene glycol or a tetraglycol furol, and water.

Regarding amounts, in one embodiment the alcohol is present in an amount of about 30 to 60% by weight of the composition, the polyalcohol is present in an amount of about 10 to 20% by weight of the composition, and the monoalkyl ether of diethylene glycol or tetraglycol furol is present in an amount between about 1 to 10% by weight of the composition. Preferably, the alcohol is present in an amount of about 40 to 55% by weight of the composition, the polyalcohol is present in an amount of about 10 to 20% and preferably 10 to 15% by weight of the composition, and the monoalkyl ether of diethylene glycol or tetraglycol furol is present in an amount between about 1 to 5% by weight of the composition.

Other useful carriers include the combination of a polyalcohol and either a monoalkyl ether of diethylene glycol or a tetraglycol furol. A preferred polyalcohol is propylene glycol. In this carrier, the relative amounts of polyalcohol to monoalkyl ether of diethylene glycol or tetraglycol furol is about 1:1 to 10:1 and preferably 2.5:1 to 7:1. The amount of polyalcohol can be from 1 to 30% by weight of the carrier, with the monoalkyl ether of diethylene glycol or tetraglycol furol being present in an amount of 1 to 30% and preferably from 2.5 to 20%. Other solvents from the types disclosed herein can be added to these carriers if desired, but it is not necessary to have more than three or four components in the carrier in addition to the water that constitutes the balance. For instance, a preferred carrier of the present invention comprises a short-chain alcohol, a polyalcohol, a monoalkyl ether of diethylene glycol or a tetraglycol furol, and water. The permeation ability of these carriers can be enhanced by the presence of urea or the urea derivatives disclosed herein.

The preferred carrier comprises an alcohol selected from the group consisting of ethanol, propanol, isopropanol, 1-butanol, 2-butanol or a mixture thereof, a polyalcohol selected from the group consisting of propylene glycol, dipropylene glycol or a mixture thereof, a monoalkyl ether of diethylene glycol selected from the group consisting of diethylene glycol monomethyl ether, diethylene glycol monoethyl ether or a mixture thereof, or a tetraglycol furol, and water, wherein the alcohol is present in an amount of about 30 to 70 percent by weight of the composition, the polyalcohol is present in an amount of about 10 to 20% by weight of the composition, and the monoalkyl ether of diethylene glycol or a tetraglycol furol, whichever is included, is present in an amount of about 1 to 10% by weight of the composition. The most preferred carrier consists essentially of these three components, with its permeation ability enhanced by the presence of a urea-containing compound such as urea or the specific urea derivatives disclosed herein.

The composition may be applied directly or indirectly to the skin or mucosal surfaces. Preferably, the composition is non occlusive. The phrase “non-occlusive” as used herein refers to a system that does not trap nor segregate the skin from the atmosphere.

The composition of the invention can be in a variety of forms suitable for transdermal or transmucosal administration. For purpose of illustration and not limitation, the various possible forms for the present composition include gels, ointments, creams, lotions, microspheres, liposomes, micelles, foams, lacquers, non-occlusive transdermal patches, bandages, or dressings, or combinations thereof. Alternatively, the composition may be in the form of a spray, aerosol, solution, emulsion, nanosphere, microcapsule, nanocapsule, as well as other topical or transdermal forms known in the art. Preferably, the composition is a non-occlusive gel.

Gels are semisolid, suspension-type systems. Single-phase gels comprise macromolecules (polymers) distributed substantially uniformly throughout the carrier liquid, which is typically aqueous. However, gels preferably comprise alcohol and, optionally, an oil. Preferred polymers, also known as gelling agents, are crosslinked acrylic acid polymers, polyethylene oxides, polyoxyethylene-polyoxypropylene copolymers and polyvinylalcohol; cellulosic polymers (hydroxypropyl cellulose, hydroxyethyl cellulose, hydroxypropyl methylcellulose, hydroxypropyl methylcellulose phthalate, methyl cellulose); gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Ointments are generally semisolid preparations typically based on petrolatum or other petroleum derivatives. The phrase “semi-solid” composition means a heterogeneous system in which one solid phase is dispersed in a second liquid phase. They generally provide optimum drug delivery, and, preferably, emolliency. Oleaginous ointment bases include, for example, vegetable oils, fats obtained from animals, and semisolid hydrocarbons obtained from petroleum. Emulsifiable ointment bases contain little or no water and may comprise anhydrous lanolin or hydrophilic petrolatum. Emulsion ointment bases are either water-in-oil (W/O) emulsions or oil-in-water (O/W) emulsions, and may include, for instance, cetyl alcohol, glyceryl monostearate, lanolin and stearic acid. Preferred water-soluble ointment bases are prepared from polyethylene glycols of varying molecular weight.

Creams are generally viscous liquids or semisolid emulsions, e.g., oil-in-water or water-in-oil. Cream bases are typically water-washable, and comprise an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally comprises a humectant. The emulsifier in a cream formulation is generally a nonionic, anionic, cationic or amphoteric surfactant.

Lotions are generally defined as preparations to be applied to the skin surface without friction. They are typically liquid or semi-liquid preparations in which solid particles, including the active agent, are present in a water or alcohol base. Lotions are usually suspensions of solids, and preferably, for the present purpose, comprise a liquid oily emulsion of the oil-in-water type. Lotions are preferred for treating large body areas, due to the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, and the like.

Microspheres generally encapsulate a drug or drug-containing formulation. They are generally although not necessarily formed from lipids, preferably charged lipids such as phospholipids. Preparation of lipidic microspheres is well known in the art and described in the pertinent texts and literature.

Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic and anionic liposomes are readily available. or can be easily prepared using readily available materials such as materials include phosphatidyl choline, cholesterol, phosphatidyl ethanolamine, dioleoylphosphatidyl choline (DOPC), dioleoylphosphatidyl glycerol (DOPG), diopalmitoylphosphatidyl choline (DPPC), dipalmitoylphosphatidyl glycerol (DPPG), dioleoylphoshatidyl ethanolamine (DOPE), among others. Methods for making liposomes using these materials are well known in the art.

Micelles, as known in the art, comprise surfactant molecules arranged such that their polar headgroups form an outer spherical shell, while their hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

As mentioned above, the composition may also be in the form of a transdermal patch. Generally, transdermal patches comprise an adhesive layer or matrix comprising the a composition or formulation, a backing layer, and a protective liner attached to the adhesive layer such that the composition or formulation is covered by the liner and unexposed until the protective liner is peeled off by the patch user. Typically, the patch adhesive layer or matrix serves as the carrier for the active agent or active agents to be administered to the patch user. Alternatively, additional layers may be included between the patch adhesive or matrix layer and the backing layer to include additional active agents, or non-toxic polymers well known in the art used to carry drugs or act as rate-controlling membranes.

The composition of the invention may also comprise various additives, as known to those skilled in the art. For instance, solvents, antimicrobial agents, gelling agents, preservatives, antioxidants, buffers, humectants, sequestering agents, moisturizers, surfactants, emollients, additional permeation enhancers, opacifiers, fragrances, colorants, thickening agents, stabilizers, and the like, may be added to the composition.

For the purpose of illustration suitable solvents include, but are not limited to, ethanol, isopropanol, glycol, glycofurol, dimethyl isosorbide, diethylene glycol alkyls ethers, polyethylene glycols, and ethoxylated alcohol.

Antimicrobial agents may be added to the present invention to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

Gelling agents may include for example carbomer, carboxyethylene or polyacrylic acid such as carbomer 980 or 940 NF, 981 or 941 NF, 1382 or 1342 NF, 5984 or 934 NF, ETD 2020, 2050, 934P NF, 971P NF, 974P NF and carbomer derivatives; cellulose derivatives such as ethylcellulose, hydroxypropylmethylcellulose (HPMC), ethyl-hydroxyethylcellulose (EHEC), carboxymethylcellulose (CMC), hydroxypropylcellulose (HPC), hydroxyethylcellulose (HEC), natural gums such as arabic, xanthan, guar gums, alginates, polyvinylpyrrolidone derivatives; polyoxyethylene polyoxypropylene copolymers, etc; others like chitosan, polyvinyl alcohols, pectins, veegum grades, and the like. Other suitable gelling agents to apply the present invention include, but are not limited to, carbomers. Alternatively, other gelling agents or viscosants known by those skilled in the art may also be used. The gelling agent or thickener is present from about 0.2 to about 30% w/w depending on the type of polymer, as known by one skilled in the art.

Preservatives such as benzalkonium chloride and derivatives, benzoic acid, benzyl alcohol and derivatives, bronopol, parabens, centrimide, chlorhexidine, cresol and derivatives, imidurea, phenol, phenoxyethanol, phenylethyl alcohol, phenylmercuric salts, thimerosal, sorbic acid and derivatives. The preservative is present from about 0.01 to about 10% w/w depending on the type of compound.

Antioxidants such as but not limited to tocopherol and derivatives, ascorbic acid and derivatives, butylated hydroxyanisole, butylated hydroxytoluene, fumaric acid, malic acid, propyl gallate, metabisulfites and derivatives. The antioxidant is present from about 0.001 to about 5% w/w depending on the type of compound.

Buffers such as carbonate buffers, citrate buffers, phosphate buffers, acetate buffers, hydrochloric acid, lactic acid, tartaric acid, diethylamine, triethylamine, diisopropylamine, aminomethylamine. Although other buffers as known in the art may be included. The buffer may replace up to 100% of the water amount within the composition.

Humectants such as glycerin, propylene, glycol, sorbitol. The humectant is present from about 1 to 10% w/w depending on the type of compound.

Sequestering agents such as edetic acid. The sequestering agent is present from about 0.001 to about 5% w/w depending on the type of compound.

Moisturizer such as docusate sodium, polyoxyethylene alkyl ethers, polyoxyethylene castor oil derivatives, polyoxyethylene stearates, polyoxyethylene sorbitan fatty acid esters, sodium lauryl sulfate. The moisturizer is present from about 1.0 to about 5% w/w depending on the type of compound.

Surfactants including anionic, nonionic, or cationic surfactants. The surfactant is present from about 0.1 to about 30% w/w depending on the type of compound.

Emollients such as but not limited to cetostearyl alcohol, cetyl esters wax, cholesterol, glycerin, fatty esters of glycerol, isopropyl myristate, isopropyl palmitate, lecithins, light mineral oil, mineral oil, petrolatum, lanolins, and combinations thereof. The emollient is present from about 1 to about 30% w/w depending on the type of compound.

The phrase “permeation enhancer” as used herein means an agent which improves the rate of percutaneous transport of active agents across the skin or use and delivery of active agents to organisms such as animals, whether for local application or systemic delivery. Additional permeation enhancer(s) may be incorporated in the composition, although in a preferred embodiment, urea is administered without any other permeation enhancers. Examples of suitable secondary enhancers (or “co-enhancers”) include, but are not limited to, compounds cited in “Percutaneous Penetration Enhancers”, eds. Smith et al. (CRC Press, 1995), the content of which is incorporated herein by reference.

For example, sulfoxides such as dimethylsulfoxide and decylmethylsulfoxide; surfactants such as sodium laurate, sodium lauryl sulfate, cetyltrimethylammonium bromide, benzalkonium chloride, poloxamer (231, 182, 184), tween (20, 40, 60, 80) and lecithin; the 1-substituted azacycloheptan-2-ones, particularly 1-n-dodecylcyclazacycloheptan-2-one; fatty alcohols such as lauryl alcohol, myristyl alcohol, oleyl alcohol and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate, amides and other nitrogenous compounds such as dimethylacetamide (DMA), dimethylformamide (DMF), 2-pyrrolidone, 1-methyl-2-pyrrolidone, ethanolamine, diethanolamine and triethanolamine, terpenes; alkanones, and organic acids, particularly salicylic acid and salicylates, citric acid and succinic acid.

Alternatively, other permeation enhancer(s) suitable to be used with the present invention may be known by those skilled in the art. The permeation enhancer is present from about 0.1 to about 30% w/w depending on the type of compound. Preferably the secondary permeation enhancers are fatty alcohols and fatty acids, and more preferably fatty alcohols. Preferably, the fatty alcohols have the formula the CH₃(CH₂)ₙ(CH)ₘCH₂OH wherein n ranges from (8−m) to (16−m) and m=0 to 2.

The compositions of the present invention may be manufactured by conventional techniques of drug formulation, particularly topical and transdermal drug formulation, which are within the skill of the art. Such techniques are disclosed in “Encyclopedia of Pharmaceutical Technology, 2^{nd} Ed., edited by J. Swarbrick and J. C. Boylan, Marcel Dekker, Inc., 2002, the content of which is incorporated herein by reference.

In one preferred embodiment, the invention provides a composition for the transdermal administration of an anticholinergic or antispasmodic agent, preferably oxybutynin. As pointed out above, the compositions are useful in a variety of contexts, as will be readily appreciated by those skilled in the art. For example, the preferred agent, oxybutynin has been indicated for the treatment of hyperactivity of the detrusor muscle (over activity of the bladder muscle) with frequent urge to urinate, increased urination during the night, urgent urination, involuntary urination with or without the urge to urinate (incontinence), painful or difficult urination. Generally, although not necessarily, these disorders are caused by a neurogenic bladder. See, Guittard et al., U.S. Pat. No. 5,674,895, the content of which is incorporated herein by reference. In addition, oxybutynin may treat other conditions and disorders that are responsive to transdermal administration of oxybutynin, such as detrusor hyperreflexia and detrusor instability. The other anticholinergic or antispasmodic agents have also been indicated for symptomatic treatment of overactive bladder and/or urinary incontinence. Accordingly, in another aspect of the invention, a method is provided for the treatment of overactive bladder or urge and urinary incontinence in a subject, comprising administering to a subject in need thereof, a topical or transdermal composition of the present invention.

The present invention provides for a novel topical or transdermal composition comprising a therapeutically effective anticholinergic or antispasmodic agent, in particular, a composition for enhancing the permeation or penetration of anticholinergic or antispasmodic agents across the dermal or mucosal surfaces of a mammalian subject. The term “transdermal” as used herein intends to include both transdermal (or “percutaneous”) and transmucosal administration, i.e., delivery by passage of a drug through the skin or mucosal tissue and into the bloodstream.

Generally, the composition comprises the anticholinergic or antispasmodic agent in an amount sufficient to provide a suitable daily dose to a subject in need. Accordingly, the amount of anticholinergic or antispasmodic agent in the composition may vary and will depend on a variety of factors, including the disease or condition to be treated, the nature and activity of the particular active agent, the desired effect, possible adverse reactions, the ability and speed of the active agent to reach its intended target, as well as other factors within the particular knowledge of the patient and physician.

Preferably, the amount of anticholinergic or antispasmodic agent in the composition is present at an amount between about 0.1% to about 20%, more preferably about 0.5% to about 10%, and most preferably about 1% to about 5% of the composition by weight. Preferably, the agent is oxybutynin or a pharmaceutically acceptable salt thereof.

Preferably, the urea-containing compound is present in an amount of between about 1% to 20%, more preferably about 1% to 15%, and most preferably about 1% to 10% by weight of the composition for enhancing permeation of the anticholinergic or antispasmodic agent. Preferably, the urea-containing compound is urea.

Preferably, the carrier suitable for transdermal or topical administration comprises the combination of water, an alcohol, a polyalcohol, and either a monoalkyl ether of diethylene glycol or a tetraglycol furol, wherein the alcohol is present in an amount of about 30 to 60% by weight of the composition, the polyalcohol is present in an amount of about 10 to 20% by weight of the composition, and the monoalkyl ether of diethylene glycol or tetraglycol furol is present in an amount between about 1 to 5% by weight of the composition.

The invention also provides a method for the treatment of overactive bladder or urge and urinary incontinence in a subject by administering to a subject in need thereof, a topical or transdermal composition of the present invention.

In one preferred embodiment of the invention, the daily dosage of oxybutynin is not more than 120 milligrams over a 24-hour period, and the composition is in the form of a non-occlusive gel. In one most preferred embodiment of the invention, the daily dosage of oxybutynin is not more than 90 milligrams over a 24-hour period, and the composition is in the form of a non-occlusive gel.

In a preferred embodiment, the transdermal compositions of the present invention reduce the peak plasma concentrations of oxybutynin, such that the incidence, the intensity, and combination thereof, of anticholinergic or antimuscarinic adverse drug experiences associated with oxybutynin treatment therapy is lowered, as evidenced by blood plasma concentrations. For example, the administration to a subject of a daily dose of about 30 mg to 90 mg of oxybutynin results into peak plasma concentration of oxybutynin (Cmax) and steady state plasmatic level of oxybutynin (Cavg) a ratio is 1.3 to 1.5. Similarly, the transdermal compositions of the present invention also reduce the peak plasma concentrations of oxybutynin metabolites, e.g., N-desethyloxybutynin (NDEO).

In another preferred embodiment of the invention, the daily dosage of racemic oxybutynin administered to a subject in need thereof by the means of a composition according to the present invention is between about 1 to 20 milligrams over a 24-hour period. In yet another preferred embodiment, the daily dosages of an individual enantiomer of oxybutynin is preferably lower than the corresponding racemate dose, and is about 0.5 to about 15 milligrams over a 24-hour period.

In yet another preferred embodiment, the administration to a subject of about 30 mg to 90 mg of oxybutynin by the means of a composition according to the present delivers a mean cumulative plasmatic daily dose of oxybutynin ranging from about 0.5 to about 10 mg a day, or a mean plasma area under the curve (AUC) of oxybutynin ranging from about 40 to about 500 ng*h/mL over that same period.

Advantageously, the method of the invention provides a steady plasma concentration of oxybutynin to a subject administered with the composition as well as reduces peak plasma concentrations of oxybutynin and lowers a number of incidences and/or intensities of oxybutynin-associated side effects. Preferably, the method provides a sustained transdermal oxybutynin flux allowing therapeutic levels of oxybutynin for at least 24 hours. More preferably, the method provides a sustained transdermal oxybutynin flux allowing therapeutic levels of oxybutynin for at least 48 hours. Most preferably, the method provides a sustained transdermal oxybutynin flux allowing therapeutic levels of oxybutynin for at least 72 hours. Thus, the composition only needs to be administrated every once a day, every other day, every third day or twice per week.

In a most preferred embodiment, the composition is a non-occlusive gel which may be administered once-a-day upon the abdomen, shoulder, or thigh of the subject with a preferred applied daily dose of oxybutynin of between 0.06 and 0.18 mcg per cm². A preferred daily amount of gel applied to the subject is not more than 10 g. A most preferred daily amount of gel applied to the subject is not more than 5 g. An even more preferred daily amount of gel applied to the subject is not more than 3 g.

The present method for treating overactive bladder or urge incontinence in a subject provides greater patient compliance. It has been found that the present method not only provides greater bioavailability of the drug associated with the permeation enhancer of urea-containing compound, but also provides a steady plasma drug concentration. Thus, the composition administered to the patient comprises lower amounts of drug to achieve therapeutic effects, i.e., greater bioavailability, and avoids the common peaks in plasma drug concentrations. Additionally, it has been found that when the anticholinergic drug is oxybutynin, the ratio of oxybutynin to oxybutynin metabolites ratio is higher than other oxybutynin compositions. Accordingly, the method of the invention advantageously reduces the number of incidences and/or the intensity of common undesirable side-effects associated with oxybutynin metabolites. Some said common undesirable side effects include dry mouth, accommodation disturbances, nausea and dizziness. Thus, the method of the invention will provide greater patient compliance.

Advantageously, the method of the invention provides symptomatic treatment of a number of conditions including hyperactivity of the detrusor muscles, frequent urge to urinate, decreased bladder capacity, increased urination during the night, urgent urination, involuntary urination with or without the urge to urinate (incontinence), an/or painful or difficult urination. It is to be understood that while the invention has been described in conjunction with the preferred specific embodiments thereof, the foregoing description is intended to illustrate and not to limit the scope of the invention. Other aspects, advantages and modifications will be apparent to those skilled in the art to which the invention pertains.

### EXAMPLES

The following examples are merely illustrative of the present invention and they should not be considered as limiting the scope of the invention in any way, as these examples and other equivalents thereof will become apparent to those skilled in the art in light of the present disclosure and the accompanying claims.

### Example 1

A gel composed by oxybutynin base 3.00% w/w, ethanol 54.22% w/w, purified water 17.23% w/w, diethylene glycol monoethyl ether 2.50% w/w, propylene glycol 15.0% w/w, hydroxypropylcellulose (KLUCEL™ MF Pharm) 2.00% w/w, butylhydroxytoluene (BHT) 0.05% w/w, hydrochloric acid HCl 0.1M 6.00%, was prepared by dissolving the oxybutynin base in the ethanol/propylene glycol/diethylene glycol monoethyl ether/BHT mixture. Purified water was then added and pH adjusted with hydrochloric acid 0.1N. Hydroxypropylcellulose was then thoroughly dispersed in the hydro-alcoholic solution under mechanical stirring at room temperature at a suitable speed ensuring good homogenization of the formulation while avoiding lumps formation and air entrapment until complete swelling.

### Example 2

A gel composed by oxybutynin base 3.00% w/w, ethanol 50.72% w/w, purified water 14.73% w/w, diethylene glycol monoethyl ether 2.50% w/w, propylene glycol 15.0% w/w, urea 5.00%, hydroxypropylcellulose (KLUCEL™ MF Pharm) 2.00% w/w, butylhydroxytoluene (BHT) 0.05% w/w, hydrochloric acid HCl 0.1M 7.00%, was prepared according to manufacturing process described in Example 1.

### Example 3

A gel composed by oxybutynin base 3.00% w/w, ethanol 34.22% w/w, isopropanol 20.00% w/w, purified water 20.23% w/w, diethylene glycol monoethyl ether 2.50% w/w, propylene glycol 15.0% w/w, hydroxypropylcellulose (KLUCEL™ MF Pharm) 2.00% w/w, butylhydroxytoluene (BHT) 0.05% w/w, hydrochloric acid HCl 0.1M 3.00%, was prepared according to manufacturing process described in Example 1.

### Example 4

A gel composed by oxybutynin base 3.00% w/w, ethanol 66.50% w/w, purified water 22.39% w/w, hydroxypropylcellulose (KLUCEL™ MF Pharm) 2.00% w/w, hydrochloric acid HCl 0.1M 6.11%, was prepared according to manufacturing process described in Example 1.

### Example 5

A gel composed by oxybutynin base 3.00% w/w, ethanol 30.72% w/w, isopropanol 20.00% w/w, purified water 19.15% w/w, diethylene glycol monoethyl ether 2.50% w/w, propylene glycol 15.0% w/w, urea 5.00% w/w, hydroxypropylcellulose (KLUCEL™ MF Pharm) 2.00% w/w, butylhydroxytoluene (BHT) 0.05% w/w, hydrochloric acid HCl 0.1M 2.58%, was prepared according to manufacturing process described in Example 1.

### Example 6

A gel composed by oxybutynin base 3.00% w/w, ethanol 70.00% w/w, purified water 8.19% w/w, urea 5.00% w/w, hydroxypropylcellulose (KLUCEL™ MF Pharm) 2.00% w/w, butylhydroxytoluene (BHT) 0.05% w/w, hydrochloric acid HCl 0.1M 11.76%, was prepared according to manufacturing process described in Example 1.

### Examples 7-16

### In Vitro Comparative Studies

In vitro drug permeation and biodistribution experiments through ear pig skin were made using a Franz Vertical Diffusion Cell diffusion chamber. Cutaneous penetration studies in vitro through human skin are limited due to the lack of availability of the human skin. It is largely described in the literature that ear pig skin can be used as the closest model to human skin in the assessment of percutaneous absorption of chemicals.

Fresh cadaver ear pig skin obtained from slaughterhouses was processed according to standard operating procedures. The ears were evaluated on their integrity (no bites, scratches or redness) and condition. The skin was excised from the ears with the help of scalpels, avoiding perforations or any damage. The excised skin samples were rinsed with PBS solution and placed on a surface for successive punching of skin disks. The skin disk pieces were mounted between the sections of a vertical diffusion cell having 1.77 sqcm of surface area, the epidermal facing up. 10 or 50 mg of the transdermal devices exemplified previously was applied over the epidermal layer whilst the dermal layer contact with the receptor solution: 2.0% w/v polyoxyethylene 20 oleyl ether (Oleth 20), with PBS, pH 7.4. The receptor chamber was maintained at 35° C. and the studies were conducted under non-occlusive conditions and at 600 rpm of stirring speed. At given time points, samples were withdrawn from the receptor solution and the receptor chamber was immediately refilled with fresh solution. All samples taken from the receptor solution (permeated drug) were analyzed using a high performance liquid chromatography (HPLC) method. The total amount of drug permeated (mcg/sqcm) during the study duration and the transdermal flux (mcg/sqcm/h) were determined for each study.

All the “Drug Permeation Studies” described above, were conducted under the following conditions: Franz Vertical Diffusion Cells (Hanson Research Inc.) were used and ear pig skin was used as experimental model. The receptor solution was 2% w/w polyoxyethylene 20 oleyl ether (Oleth 20), PBS 10 mM, pH 7.4. The experiments were conducted under non-occlusive conditions, at 35° C. and 600 rpm of stirring speed. Prior to the beginning of the study, the skin pieces were mounted on the permeation cells and maintained at 35° C. in contact with the receptor solution. After loading the formulation over the skin, at the indicated times, 1 ml of the receptor solution was withdrawn, and the receptor chamber was immediately refilled with fresh solution.

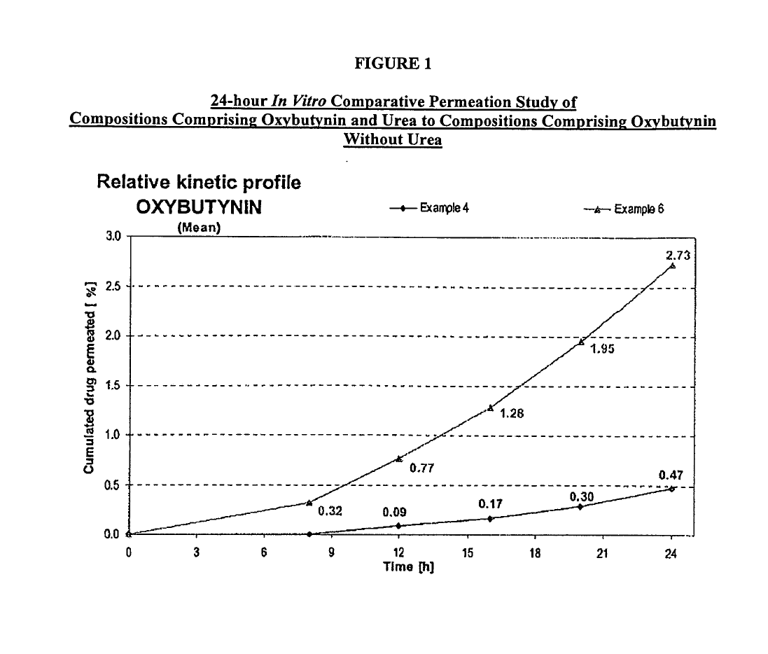

The accompanying figures represent studies which further exemplify the invention described herein. The figures are for the purpose of illustration and not for the limitation of the invention. With reference to FIG. 1, a graph is provided which demonstrates that the composition comprising urea has a 6.7-fold increase in the amount of cumulated, permeated oxybutynin after 24 hours, i.e., 2.73% for Example 6, which comprises 5% urea, versus 0.47% for Example 4 for the reference formulation, not containing urea.

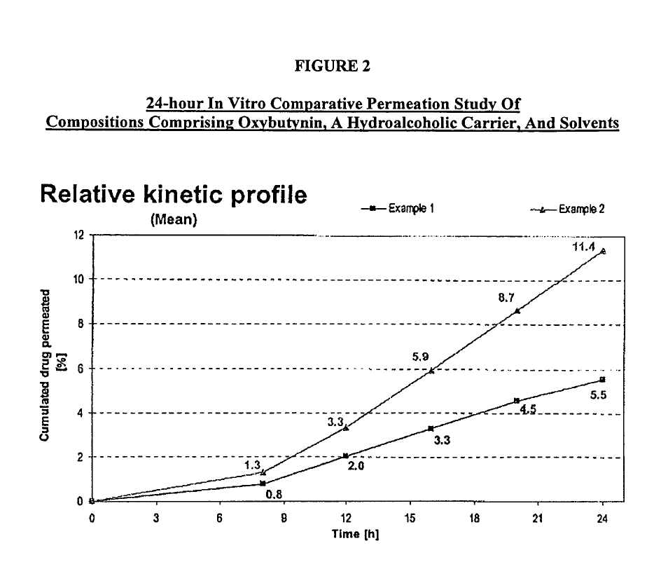

Referring to FIG. 2, a graph illustrates the results of a 24-hour in vitro comparison permeation study comparing a composition comprising oxybutynin, a hydroalcoholic carrier, additional solvents, i.e., diethylene glycol monoethyl ether and propylene glycol, and no urea, to a composition comprising oxybutynin, a hydroalcoholic carrier, additional solvents, and urea. As shown, after 24 h permeation, the amount of cumulated permeated oxybutynin is significantly higher for Example 2, which comprises urea in an amount of 5% than Example 1, the reference composition which does not contain any urea. The results show a permeation of 11.4% versus 5.5%, respectively.

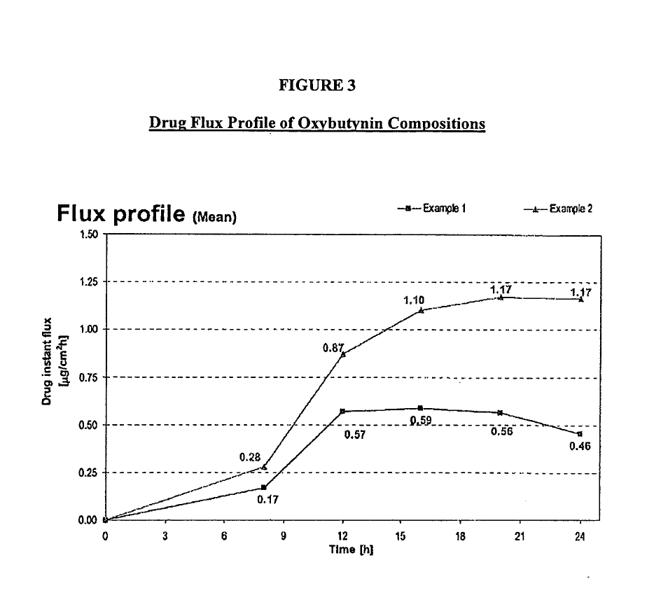

FIG. 3 illustrates that the maximal transdermal oxybutynin flux is almost 2 times higher in Example 2 than in Example 1, 1.17 μg/cm²h versus 0.59 μg/cm²h, respectively. Additionally, the results show the maximal transdermal oxybutynin flux is reached after 16 hours for Example 1, which contains no urea, and the maximal transdermal oxybutynin flux is reached after at least 20 hours in Example 2. Accordingly, the presence of urea in the composition, enhances the transdermal oxybutynin permeation, and also delays oxybutynin maximal transdermal instant flux and sustains the oxybutynin maximal transdermal instant flux. This can be responsible for sustained oxybutynin plasmatic levels in vivo after multiple application of a composition of the present invention.

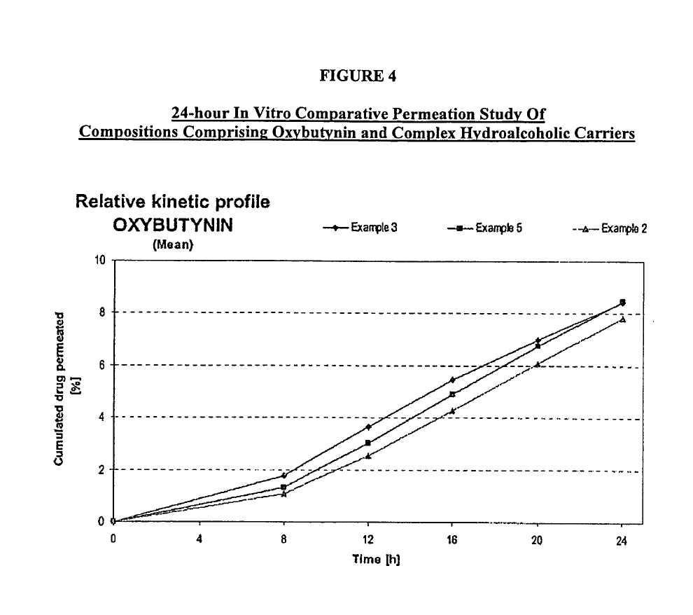

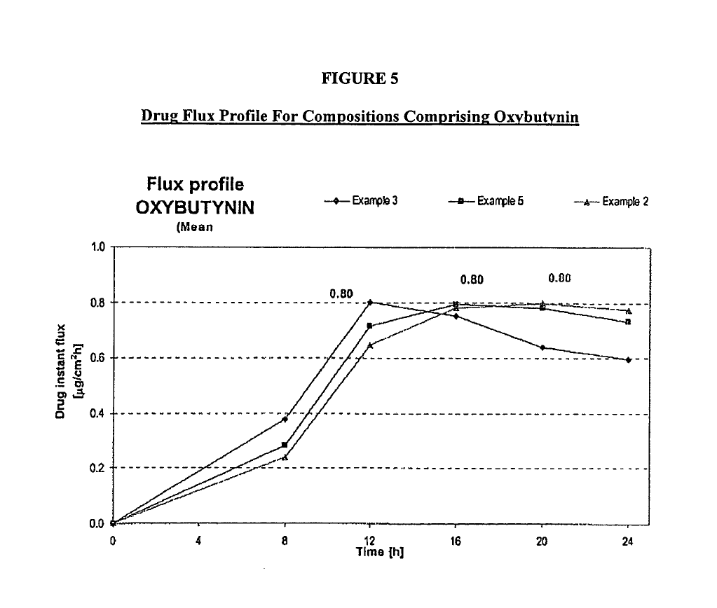

Referring now to FIG. 4, the graph illustrates the results of a permeation study comparing Examples 3, 5, and 2, described above. The relative cumulated permeated amount of oxybutynin after 24 hours is similar for each example, i.e., approximately to 8%. Now referring to FIG. 5, although the three compositions present similar oxybutynin cumulated permeated amounts as shown in FIG. 4, FIG. 5 illustrates that Examples 3, 5, and 2, have similar maximal transdermal oxybutynin flux (close to 0.80 μg/cm²h), but that the maximal transdermal oxybutynin flux is attained after 12 hours for Example 3, which contains no urea, and is attained after 16 hours for Example 5, and after 20 hours for Example 2, both of which comprise urea. Accordingly, both compositions comprising urea, Examples 2 and 5, have transdermal oxybutynin fluxes that were maintained over a longer period of time (“steady-state”).

To illustrate the superior permeation effects of urea on oxybutynin, urea was compared to two permeation enhancers well-known in the science of transdermal permeation of drugs, namely, isopropyl myristate and lauric acid. The formulations are represented below.

| Composition | Example 7 | Example 8 | Example 9 |
|---|---|---|---|
| Oxybutynin base | 3.00 | 3.00 | 3.00 |
| Urea | 5.00 | — | — |
| Isopropyl Myristate | — | 5.00 | — |
| Lauric Acid | — | — | 5.00 |
| Hydroxypropyl cellulose | 2.00 | 2.00 | 2.00 |
| Hydrochloric acid 0.1N | Q.S. pH 7.6 | Q.S. pH 7.2 | — |
| Triethanolamine | — | — | Q.S. pH 7.0 |
| Ethanol | 50.7 | 50.7 | 50.7 |
| Purified Water | Q.S. 100 | Q.S. 100 | Q.S. 100 |

Amounts are expressed as percent weight % w/w

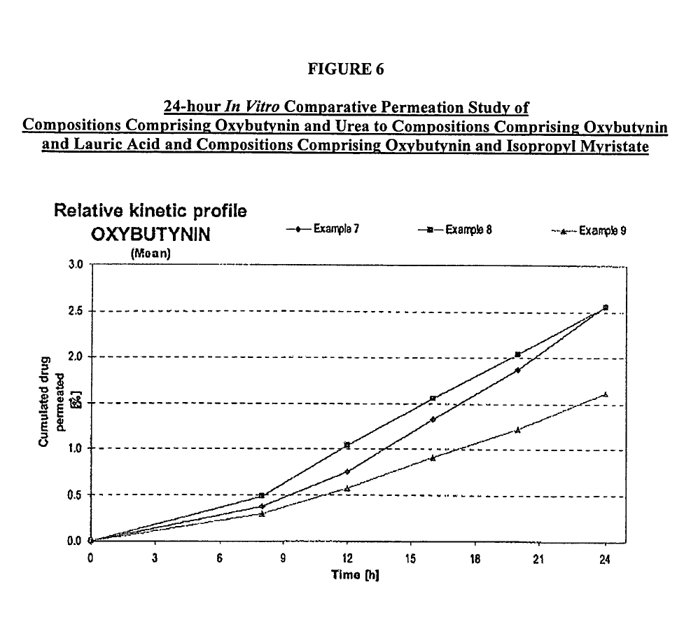

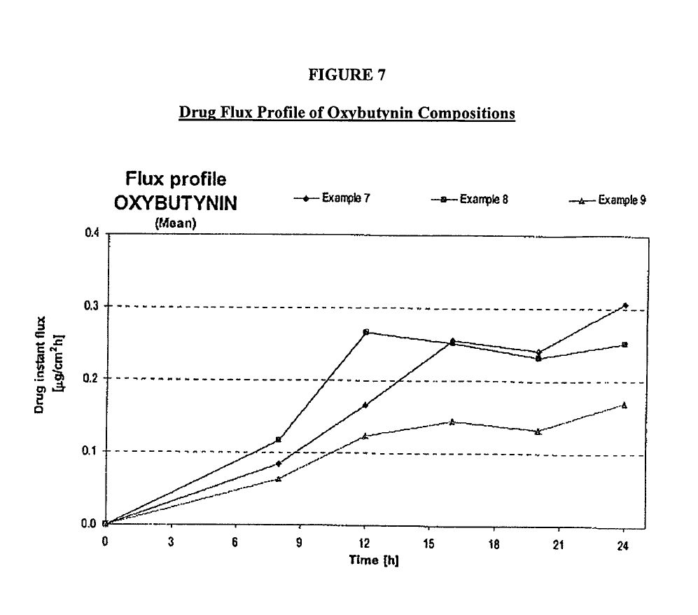

The results of the comparison study are shown in FIGS. 6 and 7. As illustrated by the graph in FIG. 6, the formulation containing urea enhances permeation of the oxybutynin as compared to lauric acid by 63%. Also illustrated in FIG. 7 is that the formulation containing isopropyl myristate exhibits cumulative oxybutynin permeation amounts similar to that of the urea formulation. However, the formulation containing the isopropyl myristate was found to be pharmaceutically undesirable due to instability of the formulation. The necessary amounts of the isopropyl myristate are difficult to dissolve in the hydroalcoholic vehicle of the invention. Accordingly, the isopropyl myristate exhibited a rapid and extensive phase separation, a phenomenon known as coalescence, within only hours.

As shown in FIG. 7, the flux profile graph indicates that the maximal flux is higher for the formulation containing urea than for the formulations containing lauric acid and isopropyl myristate by 80% and 22%, respectively. Further, the maximal flux is not reached after 24 hours for the lauric acid formulation, and is reached after 12 hours for the isopropyl myristate formulation. In sum, as illustrated by the higher permeation amounts and the higher maximal flux, urea is a superior permeation enhancer for oxybutynin than is lauric acid. Further, as illustrated by the higher maximal flux and the superior physical stability, urea is a superior permeation enhancer for oxybutynin than is isopropyl myristate.

The composition in accordance with the present invention was also compared to two other formulations containing other permeation enhancers known in the art of transdermal delivery, namely, triacetin and glycerol monooleate. Triacetin is being claimed as a permeation enhancer for oxybutynin in many references, eg U.S. Pat. No. 7,179,483. Glycerol monooleate is being claimed as a permeation enhancer for oxybutynin in U.S. Pat. No. 5,900,250. The comparative formulations are represented below.

| Composition | Example 7 | Example 10 | Example 11 |
|---|---|---|---|
| Oxybutynin base | 3.00 | 3.00 | 3.00 |
| Urea | 5.00 | — | — |
| Triacetin | — | 5.00 | — |
| Glycerol monooleate | — | — | 5.00 |
| Hydroxypropyl cellulose | 2.00 | 2.00 | 2.00 |
| Hydrochloric acid 0.1N | Q.S. pH 7.6 | Q.S. pH 7.2 | — |
| Triethanolamine | — | — | Q.S. pH 7.0 |
| Ethanol | 50.7 | 50.7 | 50.7 |
| Purified Water | Q.S. 100 | Q.S. 100 | Q.S. 100 |

Amounts are represented as percent weight by weight % w/w

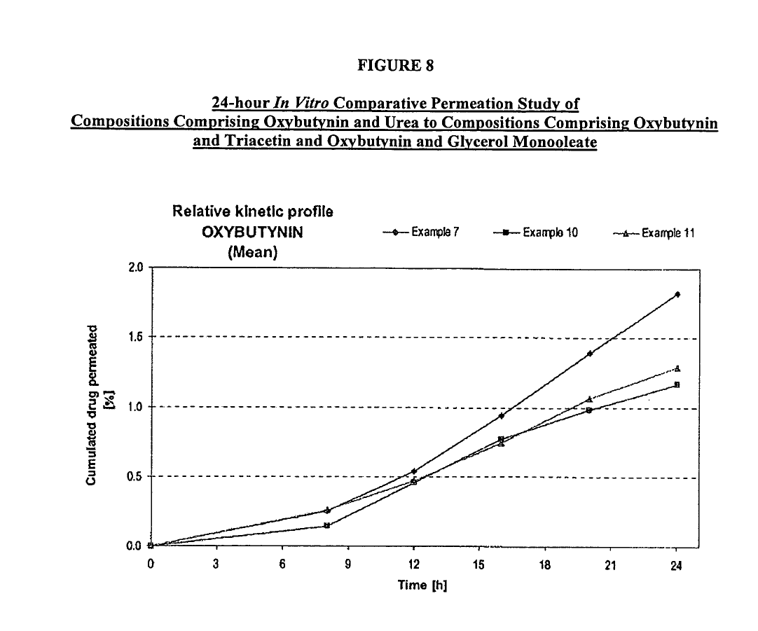

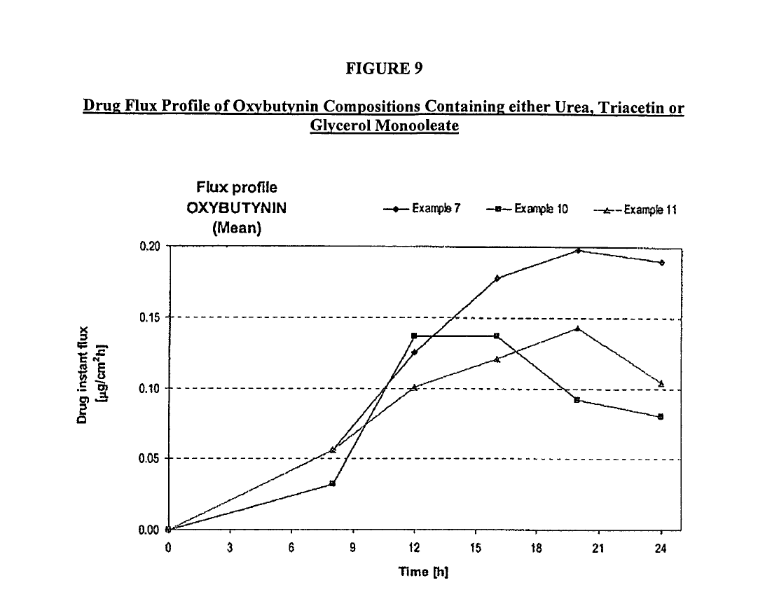

The results of this comparative study are shown in FIGS. 8 and 9. Referring to FIG. 8, the composition containing urea has increased absolute transdermal absorption of oxybutynin as compared to the formulation containing triacetin and to the formulation containing glycerol monooleate, by 38% and 57%, respectively. The relative transdermal absorption after 24 hours is also increased for the composition containing urea as compared to the formulation containing triacetin and the formulation containing glycerol monooleate, by 41% and 56%, respectively.

Referring now to FIG. 9, the maximal and steady-state fluxes are also higher for the composition containing urea. The steady-state flux for the composition containing urea is 90% higher than the formulation containing triacetin and the formulation containing glycerol monooleate. Further, the maximum flux is reached at 20-hours, as for the glycerol monooleate, but 8-hours later than the formulation containing triacetin. As illustrated, the triacetin formulation reaches its maximum flux at 12-hours. This comparison shows that the oxybutynin sustained release potential is higher for formulations comprising urea than for formulations comprising glycerol monooleate since between 20 hours and 24-hours, the maximum flux decreases by 27% for glycerol monooleate (0.104 ug/cm²h at 24 h vs. 0.143 ug/cm²h at 20 h) and only by 4% for urea (0.190 ug/cm²h at 24 h vs. 0.198 ug/cm²h at 20 h). Thus, this study illustrates that urea is a better permeation enhancer than either glycerol monooleate or triacetin as demonstrated by the higher 24-hour cumulative oxybutynin permeated amounts and the higher maximal flux. Moreover, the composition containing urea exhibits a sustained steady-state that might be responsible in vivo for lower variations in oxybutynin blood levels, and consequently for lower occurrences of undesirable side effects.

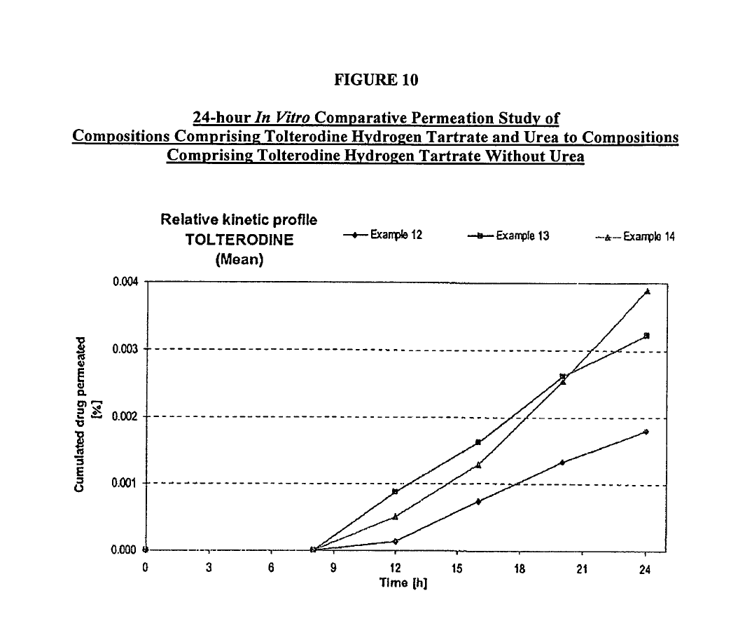

In addition to the superior permeation effects of urea for oxybutynin, FIG. 10 illustrates that the addition of urea, either alone or in the presence of other co-solvents, into a simple hydroalcoholic formulation enhances the permeation of anticholinergic agents other than oxybutynin. In this study, the effect of urea as a permeation enhancer of tolterodine hydrogen tartrate was investigated. The comparative formulations are represented below.

| Composition | Example 12 | Example 13 | Example 14 |
|---|---|---|---|
| Tolterodine hydrogen | 3.00 | 3.00 | 3.00 |
| tartrate base | | | |
| Ethanol | 40.0 | 40.0 | 40.0 |
| Urea | — | 5.00 | 5.00 |
| Propylene glycol | — | — | 15.0 |
| Diethylene glycol | — | — | 2.50 |
| monoethyl ether | | | |
| Purified water | Q.S. 100 | Q.S. 100 | Q.S. 100 |

Amounts are represented as percent weight by weight % w/w

The results of the comparison study are shown in FIG. 10. As illustrated in FIG. 10, the composition comprising urea enhances permeation of the tolterodine as compared to the reference formulation, which does not contain urea, by 85% after 24 hours. Additionally and as shown in FIG. 10, the composition comprising urea and co-solvents, 2.5% diethylene glycol monoethyl ether and 15% propylene glycol, further increases skin permeation of tolterodine by 19%. Accordingly, this study demonstrates that the addition of urea alone or in the presence of co-solvents allows enhanced permeation to other anticholinergic agents.

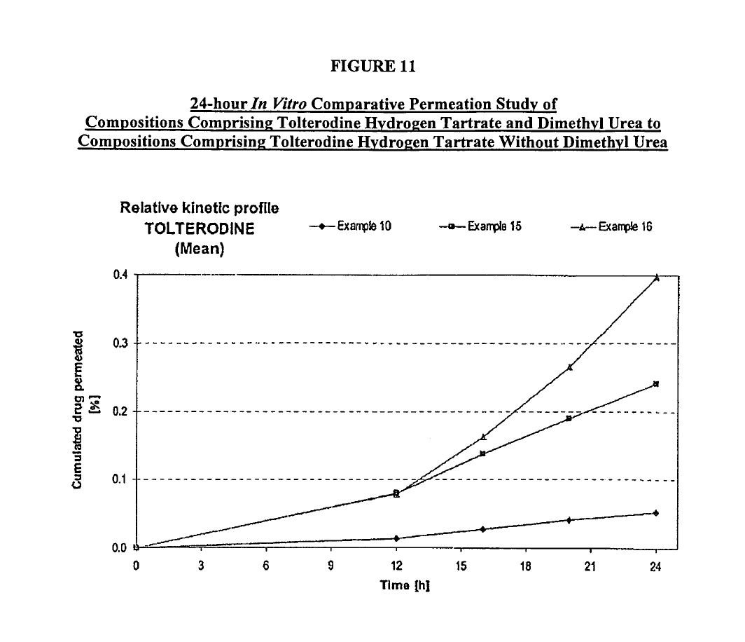

In addition to the superior permeation effects of urea on anticholinergic agents including oxybutynin and tolterodine, FIG. 11 illustrates that the addition of a urea-containing derivative, such as dimethyl urea, into a simple hydro-alcoholic formulation of tolterodine provides superior skin permeation of the drug. The comparative formulations are represented below.

| Composition | Example 10 | Example 15 | Example 16 |
|---|---|---|---|
| Tolterodine hydrogen tartrate | 3.00 | 3.00 | 3.00 |
| (expressed as a base) | | | |
| Ethanol | 40.0 | 40.0 | 40.0 |
| Dimethylurea | — | 5.00 | 5.00 |
| Propylene glycol | — | — | 15.0 |
| Diethylene glycol monoethyl | — | — | 2.50 |
| ether | | | |
| Purified water | Q.S. 100 | Q.S. 100 | Q.S. 100 |

Amounts are represented as percent weight by weight % w/w

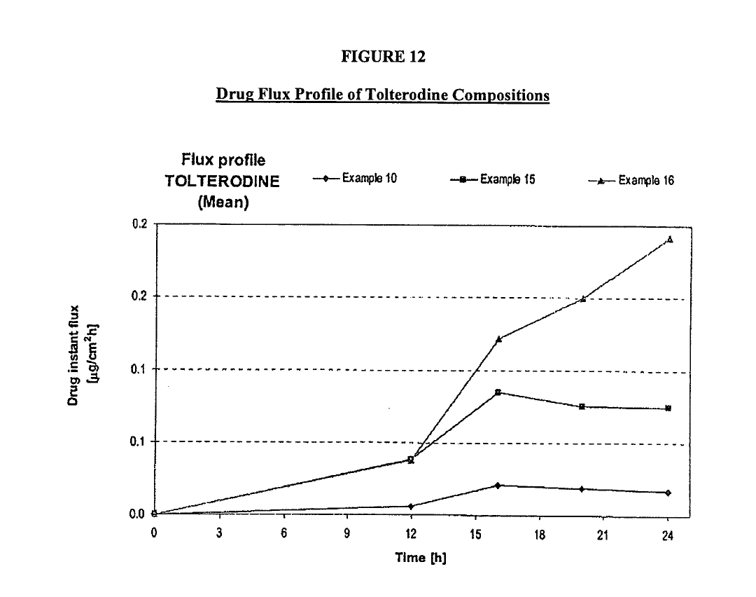

The results of the study are shown in FIGS. 11 and 12. Referring to FIG. 11, the composition comprising tolterodine and dimethyl urea enhances permeation of the tolterodine by four times as compared to the reference formulation, Example 10, which does not contain dimethyl urea. Further, the composition comprising dimethyl urea and co-solvents, propylene glycol and diethylene glycol monoethyl ether, (Example 16) further enhances permeation of the drug by 7.7 times or 66%. Accordingly, this study demonstrates that urea containing derivatives such as dimethyl urea, either alone or in the presence of co-solvents, enhance permeation of anticholinergic agents.

Referring now to FIG. 12, the composition comprising dimethyl urea enhances drug flux of tolterodine by 4 times as compared to the reference formulation. As shown, the steady state is achieved after 16 hours for each of the composition comprising dimethyl urea and the reference formulation, Example 10. However, the steady-state for the composition comprising dimethyl urea and co-solvents is not achieved even after 24 hours. In sum, this comparative study illustrates the efficacy of urea derivatives as permeation enhancers for anticholinergic agents.

### Example 17

### Pilot Pharmacokinetic Study of an Oxybutynin Gel Formulation in Healthy Volunteers

A pilot study was conducted to determine Pharmacokinetics of oxybutynin and its metabolite, N-desethyloxybutynin when administered from a composition of the present invention. The study and its results are presented below.

Healthy Caucasian females, aged 20 to 55, were recruited for the study. Subjects were required to have a body mass index of 20 to 28 kg/m² (weight in kilograms divided by the square of height in meters), to be non-smokers and to have no history of chronic medical illness or alcohol or drug abuse. Subjects were excluded on the basis of any preexisting condition or finding on the prestudy examination that would place them at risk during the study. Written informed consent was obtained from each subject before participating in any study-related procedures after discussion and explanation of the study.

Treatments were administered according to an open-label, multiple-dose, escalating dose titration pilot pharmacokinetic study and included a transdermal oxybutynin gel. Steady-state conditions were achieved by administering daily doses for 7 days. Subjects participated in 2 study periods during which the test medication (a transdermal gel) was applied once daily. 2 g of the gel (corresponding to a dose of 60 mg of oxybutynin) was applied daily during the first study period (Treatment A), then 1 g (corresponding to a dose of 30 mg of oxybutynin) during the second study period (Treatment B). A wash-out period of 7 days was observed between the two study periods. Both dosages were tested on same subjects. The design allows comparison between different dosages of the same formulation within the subject and removes inter-subject variability. The oxybutynin gels administered in this study comprised oxybutynin base 3.00% w/w, diethylene glycol monoethyl ether 2.50% w/w, propylene glycol 15.0% w/w, urea 5.00% w/w, ethanol 50.7% w/w, hydropropylcellulose KLUCEL HF 2.00% w/w, hydrochloric acid 0.1N 8.50% w/w, butylhydroxytoluene 0.05% w/w, and purified water qs.

The main objective of this study was to assess the pharmacokinetic parameters of an oxybutynin gel formulation, administered in two different doses, administering the product in 8 healthy female volunteers. Data on substances concentration at peak (Cₘₐₓ), time to reach peak (tₘₐₓ) and area under the concentration/time curve (AUC) were calculated for both oxybutynin and N-desethyloxybutynin.

The secondary objective of this study was to record safety parameters such as adverse events, skin tolerance, vital signs, e.g., blood pressure and heart rate.

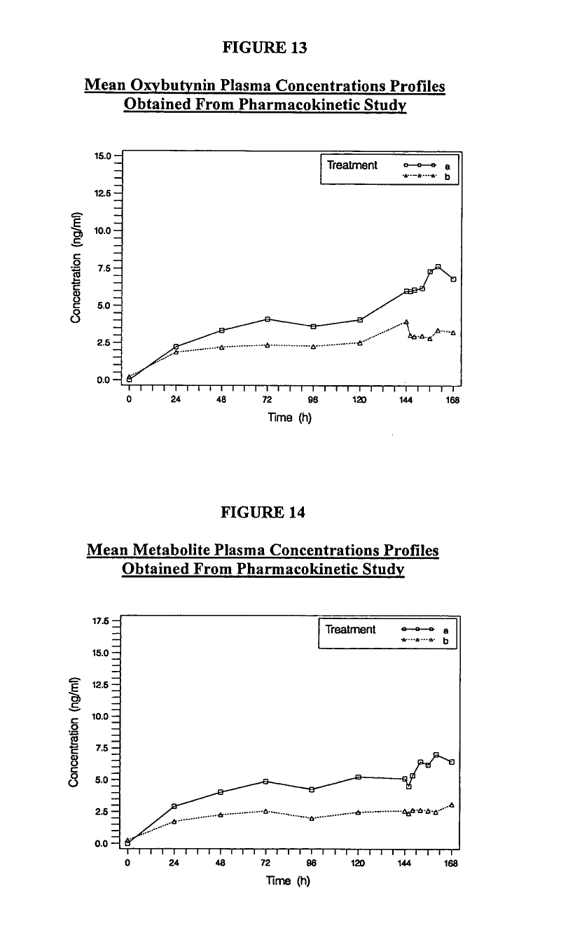

With reference to FIG. 13, the graph illustrates the mean oxybutynin plasma concentration profile from treatment A and treatment B. As can be seen between 24 hours and 168 hours, diminution of the oxybutynin dose by half, i.e., from 2 g to 1 g of gel, resulted in a 1.93-fold reduction (SD 1.08) of the mean oxybutynin plasmatic levels, i.e., from about 4.3 ng/ml (SD 2.6) to about 2.6 ng/ml (SD 1.9). As seen, an almost linear relationship between the applied oxybutynin dosage and the resulting oxybutynin plasmatic levels exists. In this regard, Tables 10, 11, and 12 show the plasmatic ratios of oxybutynin: metabolite for treatments A and B, the plasmatic ratios of oxybutynin treatment A: oxybutynin treatment B, and the plasmatic ratios of metabolite treatment A to metabolite treatment B, respectively, taken at individual sampling times.

Referring now to FIG. 14, the graph illustrates the N-desethyloxybutynin plasma concentration profile from treatment A and treatment B. Similarly to FIG. 13, diminution of the oxybutynin dose by half, i.e., from 2 g to 1 g gel, resulted in a 2.06-fold reduction (SD 1.17) of mean N-desethyloxybutynin plasmatic levels, i.e., from about 4.7 ng/ml (SD 3.3) to about 2.4 ng/ml (SD 1.2). Thus, an almost linear relationship between oxybutynin dose applied and resulting N-desethyloxybutynin plasmatic levels exists.

The reduction of the daily dose of oxybutynin resulted in a lower variability of mean plasmatic oxybutynin and mean plasmatic N-desethyloxybutynin levels all through the duration of the studies (7 days each). It is also remarkable how outstandingly “flat” the profile of N-desethyloxybutynin obtained with treatment B (corresponding to a 30 mg daily dose of oxybutynin) is and how low the N-desethyloxybutynin mean plasmatic concentrations are. Consequently, the compositions and methods provide reduced incidences of oxybutynin-associated side effects, or provide lower-intensity oxybutynin-associated side effects.

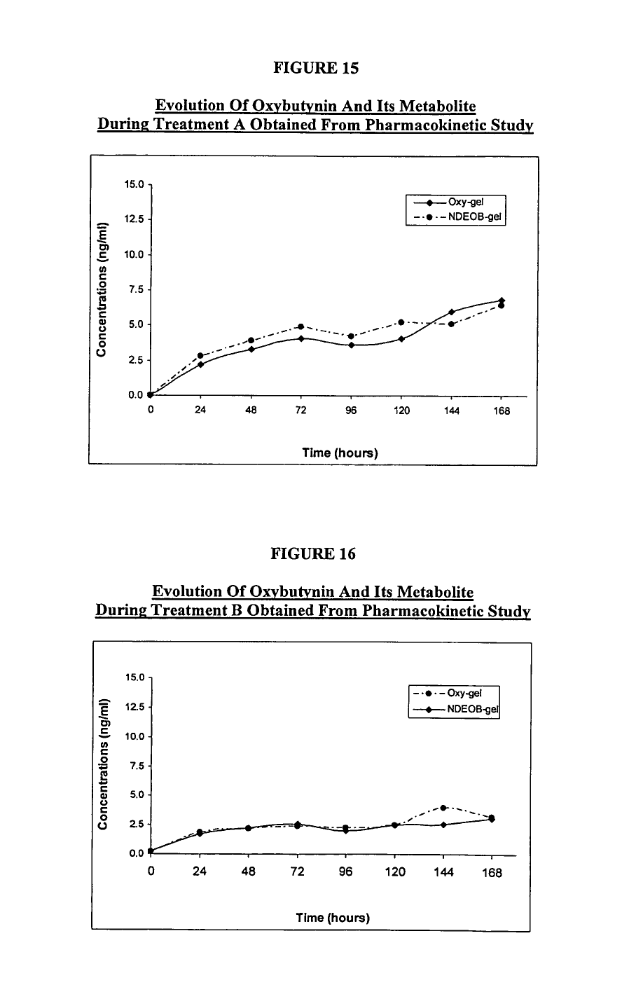

FIG. 15 shows the evolution of oxybutynin and N-desethyloxybutynin during treatment A. As illustrated in Table 3 below, the ratio of mean oxybutynin plasmatic concentrations to mean plasmatic concentration of N-Desethyloxybutynin is constant and close to 1 (Mean 1.10; SD 0.67). In this regard, Tables 6 and 8 show the oxybutynin concentrations for treatment A, and metabolite concentrations for treatment A, respectively, at individual sampling times.

TABLE 3
Oxybutynin: N-Desethyloxybutynin mean plasmatic concentrations ratio
(Treatment A)
Scheduled time

| 0 | 24 | 48 | 72 | 96 | 120 | 144 | 168 |
|---|---|---|---|---|---|---|---|
| — | 0.85 | 0.98 | 0.96 | 0.99 | 0.91 | 1.54 | 1.49 |

With reference to FIG. 16, a graph demonstrates the evolution of oxybutynin and its metabolite, N-desethyloxybutynin during treatment B. Similarly to treatment A, and as shown in Tables 5 and 6, below, the ratio of mean oxybutynin plasmatic concentrations to mean plasmatic concentration of N-Desethyloxybutynin is constant and close to 1 (Mean 1.14; SD 0.57).

TABLE 4
Oxybutynin: N-Desethyloxybutynin mean plasmatic concentrations ratio
(Treatment B)
Scheduled time

| 0 | 24 | 48 | 72 | 96 | 120 | 144 | 168 |
|---|---|---|---|---|---|---|---|
| — | 1.15 | 1.03 | 0.93 | 1.18 | 0.99 | 1.58 | 1.12 |

Accordingly, the reduction of the oxybutynin daily dose resulted in a higher and less variable ratio of mean oxybutynin to N-desethyloxybutynin mean plasmatic concentrations all through the duration of the studies (7 days each). In this regard, Tables 7 and 9 show oxybutynin concentrations for treatment B, and metabolite concentrations for treatment B, respectively, at individual sampling times.

In conclusion, the obtained oxybutynin:N-desethyloxybutynin ratios were much higher for the oxybutynin gels administered in both treatments than the ratios associated with oral administration of oxybutynin. See, Zobrist et al, Mayo Clin Proc, June 2003, Vol 78, which is incorporated herein by reference. Thus, the higher ratio provided by the transdermal administration are believed to be responsible for the fewer incidences of oxybutynin-associated side effects and/or for the lower-intensity oxybutynin-associated side effects.

Furthermore, the obtained oxybutynin:N-desethyloxybutynin ratios for the oxybutynin gels are comparable or even higher than the ratios obtained after administration of oxybutynin by a matrix-type transdermal system, as known in the art and as shown in Table 5 below. See, Zobrist et al, Mayo Clin Proc, June 2003, Vol 78, the content of which is incorporated herein by reference.

TABLE 5
Estimated Oxybutynin: N-Desethyloxybutynin mean plasmatic
concentrations ratio (OXYTROL ™ patch)
Scheduled time (hours)

| 0 | 84 | 96 | 108 | 120 | 132 | 144 | 156 | 168 | 180 |
|---|---|---|---|---|---|---|---|---|---|
| — | 0.70 | 0.84 | 0.77 | 0.80 | 0.77 | 0.75 | 0.72 | 0.75 | 0.74 |

### Example 18

### Dose-Ranging Pharmacokinetic Study of Transdermal Oxybutynin Gel for Overactive Bladder in Healthy Volunteers

The study was an open-label, randomized, parallel, three-treatment, dose-ranging pharmacokinetic study. This study was performed in 49 healthy volunteers (48 Caucasians and 1 Negroid), with 16 subjects (75% female) in each treatment arm (N=17 for adverse events assessment in treatment A). The subjects were aged 19 to 53 (mean 35) years.

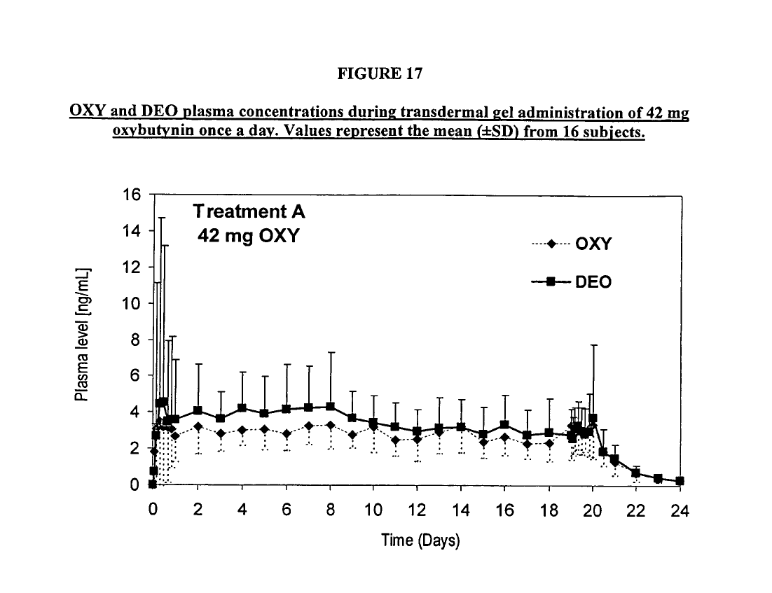

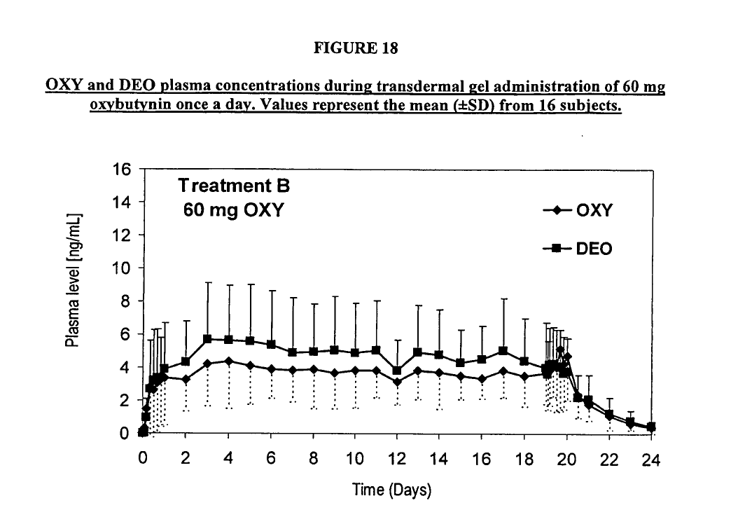

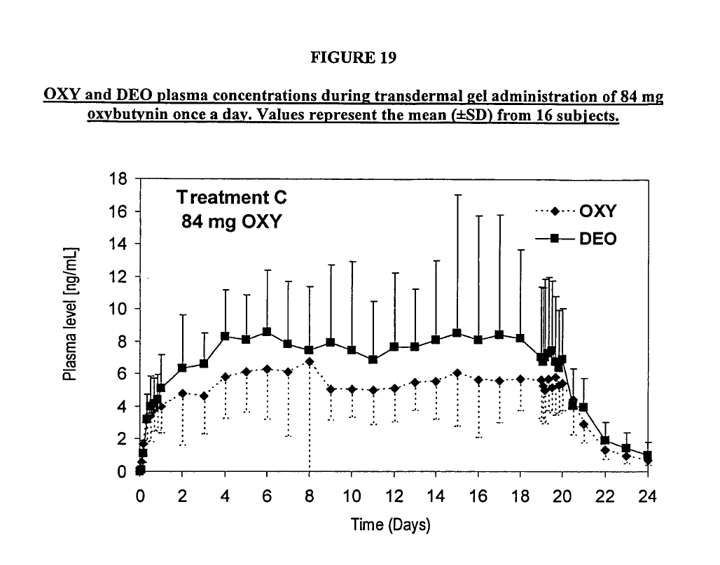

The oxybutynin transdermal gel formulation contained 3% w/w of oxybutynin base in a hydroalcoholic non-occlusive matrix including the unique combination of permeation enhancers of the present invention. Three different doses of the gel were administered topically once a day during 20 consecutive days: treatment A consisted in a daily amount of 1.4 g applied gel, corresponding to 42 mg oxybutynin; treatment B consisted in a daily amount of 2.0 g applied gel, corresponding to 60 mg oxybutynin; and treatment C consisted in a daily amount of 2.8 g applied gel, corresponding to 84 mg oxybutynin. The gel was applied daily for 20 days onto the lower abdominal region over an area of, respectively, 350 cm² for treatment A, 500 cm² for B, and 700 cm² for C. Venous blood samples for analysis of plasma oxybutynin (OXY) and N-desethyloxybutynin (DEO) levels were collected before gel application once daily up to day 26 (decay), and intensively on days 2 and 21 (8 samplings). Plasma concentrations were analyzed by means of a validated LC-MS/MS method. The pharmacokinetic parameters obtained in the present study after multiple-dose oxybutynin administration with a formulation of the present invention investigational formulation are reported on Table 13. FIGS. 17, 18 and 19 show the plasma profiles of OXY and DEO for treatment A, treatment B, and treatment C, respectively. The average DEO levels are 0.9 to 1.3 times that of the parent compound. This clinical study therefore demonstrates that topical daily administration of a transdermal gel composition of oxybutynin according to the present invention can deliver oxybutynin at therapeutic levels.

### Example 19

### Single-Dose Pharmacokinetic Study of Transdermal Oxybutynin Gel for Overactive Bladder in Healthy Volunteers

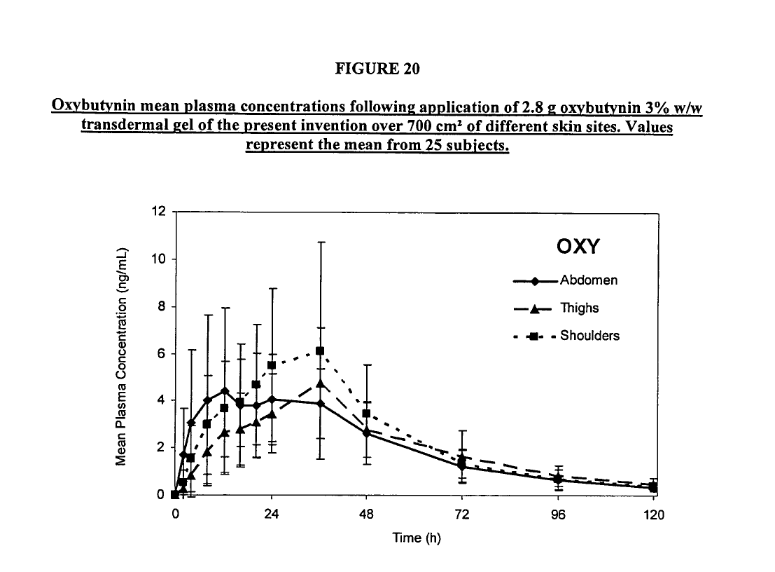

The study was an open-label, randomized, comparative three-way, application site cross-over pharmacokinetic study. This study was performed in 25 healthy volunteers (Asian Indians, 80% female). The subjects were aged 18 to 45 (mean 31) years. One dose of 2.8 g of 3% w/w oxybutynin transdermal gel of the present invention (corresponding to a does of 84 mg oxybutynin) was administered topically as a single dose on three different skin sites (700 cm²), alternatively, with 10 days washout between the doses: (A) abdomen (A); (B) thighs; or (C) shoulders. Subjects were dosed after an overnight fast of at least 10 hours. Venous blood samples for analysis of plasma OXY and the metabolite N-desethyloxybutynin (DEO) levels were collected before gel application (pre-dose), and up to 5 days post-dose (2, 4, 8, 12, 16, 20, 24, 36, 48, 72, 96, and 120 hours). Plasma concentrations were analyzed by means of a validated LC-MS/MS method. The pharmacokinetic parameters obtained in the present study after single-dose oxybutynin administration with the investigational formulation are reported in Table 14. FIG. 20 depicts the plasma profiles of OXY and DEO following application of the gel at different skin sites. The formulation of the present invention is clearly advantageous over formulations described in the anterior art thanks ton its ability to outstandingly maintain the transdermal flux close to its maximum value for a longer period of time. For instance, therapeutic plasmatic levels of oxybutynin of about 3 to about 4 ng/ml are reached within less than 6 hours and are sustained up to at least 36 hours following single-dose application of a composition of the present invention. Further, therapeutic plasmatic levels of oxybutynin of about 2 to 4 ng/ml are reached within less than about 4 hours and are sustained up to at least 48 hours following single-dose application of a composition of the present invention. Even further, therapeutic plasmatic levels of oxybutynin of about 1 to 4 ng/ml are reached after about 2 hours and are sustained up to at least 72 hours following single-dose application of a composition of the present invention (FIG. 20).

### Example 20

### Pharmacokinetic Study of R/S OXY and DEO Enantiomers in Transdermal Oxybutynin Gel for Overactive Bladder

Ten healthy volunteers (Asian Indians, 60% female) aged 18 to 35 (mean 29) years old were treated with 2.8 g of a formulation of the present invention containing 3% w/w of OXY base (84 mg OXY), administered topically as a single dose on the abdomen (700 cm²).

Subjects were dosed after an overnight fast of at least 10 hours. Venous blood samples for analysis of plasma OXY and DEO levels were collected before gel application (pre-dose), and up to 5 days post-dose (2, 4, 8, 12, 16, 20, 24, 36, 48, 72, 96, and 120 hours). Plasma concentrations were analyzed by means of a validated LC-MS/MS method.

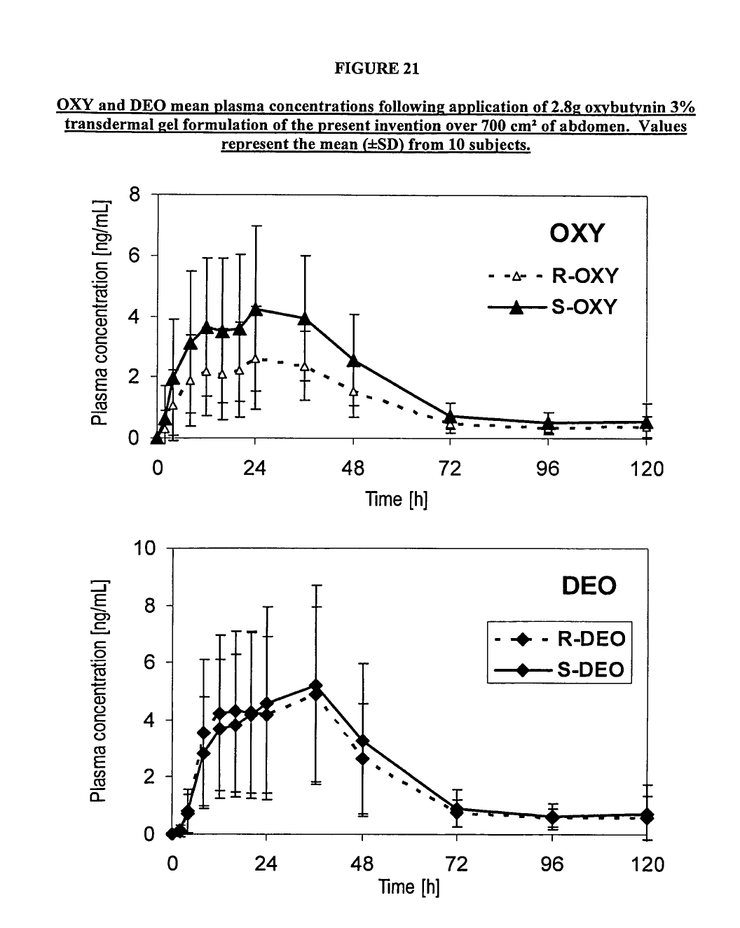

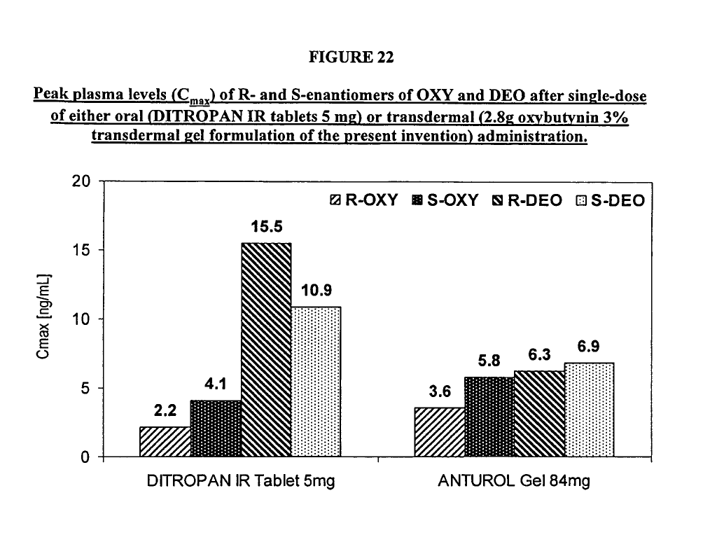

The peak plasma levels Cₘₐₓ and exposure AUC₀₋₁₂₀ obtained in the present study after single-dose OXY administration with the investigational formulation are re-ported on Table 15. As shown in FIG. 21 and Table 15, both rate and extent of absorption for R-DEO (Cₘₐₓ and AUC₀₋₁₂₀) are similar as S-DEO. This suggests that no selective metabolism occurred for the R-enantiomer upon transdermal absorption. In contrast, a single dose of DITROPAN IR 5 mg tablets (Janssen Ortho, Inc.) results in R-DEO Cₘₐₓ levels about 50% higher than S-DEO Cₘₐₓ levels (FIG. 22).

Moreover, Table 15 shows that R-DEO Cₘₐₓ levels are only 1.8-fold those of R-OXY Cₘₐₓ levels (6.3 ng/mL versus 3.6 ng/mL), whereas a single dose of DITROPAN IR 5 mg tablets (Janssen Ortho, Inc.) results in R-DEO Cₘₐₓ levels more than 7 times higher than R-OXY Cₘₐₓ levels (FIG. 22). This supports the lack of selectivity of transdermal delivery with respect to the conversion of R-enantiomers, which may potentially result in lower incidence of anticholinergic adverse events such as dry mouth, compared to the oral treatment. In fact, among the ten tested subjects, only one experienced dry mouth, which was mild in intensity.

These data show that transdermal OXY transdermal gel delivery results in substantially lower circulating R-DEO levels than oral delivery, thereby minimizing the cholinergic adverse events such as dry mouth.

It is to be understood that the above-described examples are only illustrative of preferred embodiments of the present invention. Numerous modifications and alternative arrangements may be devised by those skilled in the art without departing from the spirit and scope of the present invention and the appended claims are intended to cover such modifications and arrangements.

TABLE 6
Oxybutynin - Concentrations by Sampling Times [ng/ml], Treatment A

| | Scheduled time [h] | | | | | | | | | | | | |
|---|---|---|---|---|---|---|---|---|---|---|---|---|---|
| Subject | 0 | 24 | 48 | 72 | 96 | 120 | 144 | 146 | 148 | 152 | 156 | 160 | 168 |
| 001 | 0.000 | 4.563 | 3.211 | 3.825 | 3.872 | 4.015 | 4.291 | 2.895 | 3.979 | 4.615 | 3.891 | 5.546 | 4.871 |
| 002 | 0.000 | 1.502 | 3.137 | 4.564 | 6.429 | 5.281 | 7.730 | 7.998 | 6.861 | 6.979 | 13.821 | 7.713 | 10.703 |
| 003 | 0.000 | 2.367 | 2.673 | 2.776 | 2.310 | 2.911 | 4.396 | 3.739 | 3.727 | 3.640 | 3.890 | 3.954 | 3.056 |
| 004 | 0.000 | 1.504 | 1.971 | 4.548 | 2.719 | 2.716 | 7.509 | 8.595 | 5.862 | 4.495 | 4.432 | 3.962 | 5.569 |
| 005 | 0.000 | 3.077 | 3.237 | 5.101 | 4.134 | 5.490 | 10.751 | 9.006 | 8.812 | 9.025 | 7.702 | 13.620 | 5.859 |
| 006 | 0.000 | 0.851 | 1.187 | 1.169 | 1.417 | 1.683 | 4.450 | 6.139 | 5.676 | 3.431 | 8.174 | 8.451 | 9.652 |
| 007 | 0.000 | 2.549 | 9.033 | 8.309 | 6.015 | 7.782 | 5.968 | 5.898 | 8.051 | 9.422 | 10.205 | 11.015 | 10.388 |
| 008 | 0.000 | 1.353 | 2.184 | 2.556 | 2.083 | 2.696 | 3.170 | 3.790 | 5.836 | 8.085 | 6.710 | 7.111 | 4.702 |
| N | 8 | 8 | 8 | 8 | 8 | 8 | 8 | 8 | 8 | 8 | 8 | 8 | 8 |
| Mean | 0.000 | 2.221 | 3.329 | 4.106 | 3.622 | 4.072 | 6.033 | 6.008 | 6.101 | 6.212 | 7.353 | 7.672 | 6.850 |
| SD | 0.000 | 1.196 | 2.413 | 2.136 | 1.840 | 2.001 | 2.500 | 2.373 | 1.780 | 2.454 | 3.458 | 3.372 | 2.946 |
| SE | 0.000 | 0.423 | 0.853 | 0.755 | 0.651 | 0.707 | 0.884 | 0.839 | 0.629 | 0.868 | 1.222 | 1.192 | 1.042 |
| CV | — | 53.8 | 72.5 | 52.0 | 50.8 | 49.1 | 41.4 | 39.5 | 29.2 | 39.5 | 47.0 | 44.0 | 43.0 |
| Min | 0.000 | 0.851 | 1.187 | 1.169 | 1.417 | 1.683 | 3.170 | 2.895 | 3.727 | 3.431 | 3.890 | 3.954 | 3.056 |
| Q1 | 0.000 | 1.428 | 2.078 | 2.666 | 2.197 | 2.706 | 4.344 | 3.765 | 4.828 | 4.068 | 4.162 | 4.754 | 4.787 |
| Med | 0.000 | 1.936 | 2.905 | 4.187 | 3.296 | 3.463 | 5.209 | 6.019 | 5.849 | 5.797 | 7.206 | 7.412 | 5.714 |
| Q3 | 0.000 | 2.813 | 3.224 | 4.833 | 5.075 | 5.386 | 7.620 | 8.297 | 7.456 | 8.555 | 9.190 | 9.733 | 10.020 |
| Max | 0.000 | 4.563 | 9.033 | 8.309 | 6.429 | 7.782 | 10.751 | 9.006 | 8.812 | 9.422 | 13.821 | 13.620 | 10.703 |
| GeoM | — | 1.963 | 2.822 | 3.595 | 3.217 | 3.664 | 5.617 | 5.565 | 5.866 | 5.778 | 6.687 | 7.041 | 6.290 |
| G_CV | — | 57.4 | 63.1 | 63.7 | 57.0 | 52.7 | 41.8 | 45.1 | 31.1 | 43.0 | 49.3 | 47.0 | 47.2 |
| N\|x > 0 | 0 | 8 | 8 | 8 | 8 | 8 | 8 | 8 | 8 | 8 | 8 | 8 | 8 |

TABLE 7
Oxybutynin - Concentrations by Sampling Times [ng/ml], Treatment B

| | Scheduled time [h] | | | | | | | | | | | | |
|---|---|---|---|---|---|---|---|---|---|---|---|---|---|
| Subject | 0 | 24 | 48 | 72 | 96 | 120 | 144 | 146 | 148 | 152 | 156 | 160 | 168 |
| 001 | 0.311 | 2.366 | 3.131 | 2.120 | 1.314 | 2.066 | 3.050 | 1.594 | 1.161 | 1.660 | 1.528 | 2.334 | 3.377 |
| 002 | 0.393 | 4.373 | 3.517 | 4.972 | 7.210 | 5.654 | 7.581 | 6.849 | 5.674 | 4.166 | 3.369 | 5.363 | 6.709 |
| 003 | 0.124 | 1.506 | 2.174 | 2.106 | 1.648 | 2.320 | 2.835 | 2.358 | 2.055 | 2.182 | 2.089 | 2.297 | 1.953 |
| 004 | 0.167 | 1.330 | 3.017 | 2.577 | 2.214 | 2.291 | 2.460 | 2.830 | 2.270 | 2.278 | 2.597 | 2.114 | 2.135 |
| 005 | 0.120 | 1.572 | 1.398 | 1.632 | 1.738 | 1.920 | 2.320 | 2.142 | 2.904 | 2.512 | 3.099 | 4.940 | 2.827 |
| 006 | 0.000 | 0.544 | 0.721 | 0.695 | 0.625 | 0.456 | 1.524 | 2.550 | 1.591 | 2.527 | 2.787 | 2.304 | 1.396 |
| 007 | 0.316 | 2.559 | 2.698 | 3.238 | 1.871 | 4.304 | 9.831 | 3.806 | 4.697 | 5.193 | 3.846 | 4.169 | 5.310 |
| 008 | 0.156 | 0.641 | 0.982 | 1.563 | 1.632 | 1.237 | 2.167 | 2.147 | 3.241 | 3.427 | 3.379 | 3.520 | 2.269 |
| N | 8 | 8 | 8 | 8 | 8 | 8 | 8 | 8 | 8 | 8 | 8 | 8 | 8 |
| Mean | 0.198 | 1.861 | 2.205 | 2.363 | 2.282 | 2.531 | 3.971 | 3.035 | 2.949 | 2.993 | 2.837 | 3.380 | 3.247 |
| SD | 0.130 | 1.240 | 1.058 | 1.293 | 2.045 | 1.672 | 3.018 | 1.671 | 1.552 | 1.183 | 0.756 | 1.311 | 1.841 |
| SE | 0.046 | 0.438 | 0.374 | 0.457 | 0.723 | 0.591 | 1.067 | 0.591 | 0.549 | 0.418 | 0.267 | 0.464 | 0.651 |
| CV | 65.5 | 66.6 | 48.0 | 54.7 | 89.6 | 66.1 | 76.0 | 55.1 | 52.6 | 39.5 | 26.6 | 38.8 | 56.7 |
| Min | 0.000 | 0.544 | 0.721 | 0.695 | 0.625 | 0.456 | 1.524 | 1.594 | 1.161 | 1.660 | 1.528 | 2.114 | 1.396 |
| Q1 | 0.122 | 0.986 | 1.190 | 1.598 | 1.473 | 1.579 | 2.244 | 2.145 | 1.823 | 2.230 | 2.343 | 2.301 | 2.044 |
| Med | 0.162 | 1.539 | 2.436 | 2.113 | 1.693 | 2.179 | 2.648 | 2.454 | 2.587 | 2.520 | 2.943 | 2.927 | 2.548 |
| Q3 | 0.314 | 2.463 | 3.074 | 2.908 | 2.043 | 3.312 | 5.316 | 3.318 | 3.969 | 3.797 | 3.374 | 4.555 | 4.344 |
| Max | 0.393 | 4.373 | 3.517 | 4.972 | 7.210 | 5.654 | 9.831 | 6.849 | 5.674 | 5.193 | 3.846 | 5.363 | 6.709 |
| GeoM | 0.205 | 1.524 | 1.930 | 2.063 | 1.809 | 2.031 | 3.240 | 2.748 | 2.611 | 2.810 | 2.735 | 3.168 | 2.861 |
| G_CV | 52.1 | 79.1 | 64.7 | 63.1 | 76.2 | 89.6 | 71.6 | 46.9 | 57.2 | 38.8 | 30.8 | 39.7 | 56.5 |
| N\|x > 0 | 7 | 8 | 8 | 8 | 8 | 8 | 8 | 8 | 8 | 8 | 8 | 8 | 8 |

TABLE 8
N-desethyloxybutynin - Concentrations by Sampling Times [ng/l], Treatment A

| | Scheduled time [h] | | | | | | | | | | | | |
|---|---|---|---|---|---|---|---|---|---|---|---|---|---|
| Subject | 0 | 24 | 48 | 72 | 96 | 120 | 144 | 146 | 148 | 152 | 156 | 160 | 168 |
| 001 | 0.000 | 3.696 | 3.518 | 3.431 | 3.808 | 4.468 | 3.134 | 2.726 | 3.628 | 3.941 | 3.722 | 4.657 | 4.648 |
| 002 | 0.000 | 1.125 | 1.834 | 2.466 | 2.671 | 2.666 | 4.161 | 3.722 | 3.615 | 2.973 | 3.118 | 3.536 | 4.227 |
| 003 | 0.000 | 2.837 | 3.429 | 3.243 | 2.248 | 2.953 | 3.394 | 3.268 | 3.810 | 3.868 | 3.602 | 3.557 | 3.054 |
| 004 | 0.000 | 1.600 | 2.163 | 3.861 | 3.090 | 3.511 | 2.945 | 2.677 | 3.405 | 2.821 | 3.584 | 3.727 | 2.940 |
| 005 | 0.000 | 5.777 | 5.987 | 8.922 | 7.899 | 10.113 | 14.054 | 10.792 | 11.670 | 13.675 | 11.970 | 12.339 | 10.263 |
| 006 | 0.000 | 1.260 | 1.020 | 1.430 | 1.580 | 1.943 | 1.502 | 1.284 | 1.983 | 3.284 | 3.214 | 2.571 | 2.676 |
| 007 | 0.000 | 3.563 | 10.561 | 10.645 | 8.230 | 10.092 | 7.071 | 6.375 | 9.418 | 12.810 | 12.501 | 16.846 | 16.480 |
| 008 | 0.000 | 3.433 | 3.800 | 5.055 | 4.478 | 6.246 | 4.707 | 5.295 | 5.487 | 8.173 | 7.990 | 8.998 | 7.438 |
| N | 8 | 8 | 8 | 8 | 8 | 8 | 8 | 8 | 8 | 8 | 8 | 8 | 8 |
| Mean | 0.000 | 2.911 | 4.039 | 4.882 | 4.251 | 5.249 | 5.121 | 4.517 | 5.377 | 6.443 | 6.213 | 7.029 | 6.466 |
| SD | 0.000 | 1.566 | 3.036 | 3.233 | 2.518 | 3.265 | 3.954 | 2.992 | 3.380 | 4.533 | 4.042 | 5.198 | 4.817 |
| SE | 0.000 | 0.554 | 1.073 | 1.143 | 0.890 | 1.154 | 1.398 | 1.058 | 1.195 | 1.603 | 1.429 | 1.838 | 1.703 |
| CV | — | 53.8 | 75.2 | 66.2 | 59.2 | 62.2 | 77.2 | 66.2 | 62.9 | 70.4 | 65.1 | 73.9 | 74.5 |
| Min | 0.000 | 1.125 | 1.020 | 1.430 | 1.580 | 1.943 | 1.502 | 1.284 | 1.983 | 2.821 | 3.118 | 2.571 | 2.676 |
| Q1 | 0.000 | 1.430 | 1.999 | 2.855 | 2.460 | 2.810 | 3.040 | 2.702 | 3.510 | 3.129 | 3.399 | 3.547 | 2.997 |
| Med | 0.000 | 3.135 | 3.474 | 3.646 | 3.449 | 3.990 | 3.778 | 3.495 | 3.719 | 3.905 | 3.662 | 4.192 | 4.438 |
| Q3 | 0.000 | 3.630 | 4.894 | 6.989 | 6.189 | 8.169 | 5.889 | 5.835 | 7.453 | 10.492 | 9.980 | 10.669 | 8.851 |
| Max | 0.000 | 5.777 | 10.561 | 10.645 | 8.230 | 10.113 | 14.054 | 10.792 | 11.670 | 13.675 | 12.501 | 16.846 | 16.480 |
| GeoM | — | 2.530 | 3.226 | 4.052 | 3.664 | 4.447 | 4.171 | 3.778 | 4.609 | 5.277 | 5.248 | 5.658 | 5.269 |
| G_CV | — | 64.0 | 82.5 | 73.4 | 63.3 | 67.6 | 74.0 | 71.7 | 63.3 | 73.3 | 65.9 | 77.4 | 73.5 |
| N\|x > 0 | 0 | 8 | 8 | 8 | 8 | 8 | 8 | 8 | 8 | 8 | 8 | 8 | 8 |

TABLE 9
N-desethyloxybutynin - Concentrations by Sampling Times [ng/l], Treatment B

| | Scheduled time [h] | | | | | | | | | | | | |
|---|---|---|---|---|---|---|---|---|---|---|---|---|---|
| Subject | 0 | 24 | 48 | 72 | 96 | 120 | 144 | 146 | 148 | 152 | 156 | 160 | 168 |
| 001 | 0.284 | 1.944 | 1.892 | 2.099 | 0.698 | 1.538 | 1.843 | 1.637 | 1.307 | 1.354 | 1.692 | 1.743 | 1.930 |
| 002 | 0.375 | 1.532 | 2.071 | 3.072 | 2.844 | 3.215 | 3.184 | 2.907 | 2.548 | 2.279 | 2.224 | 1.910 | 3.017 |
| 003 | 0.105 | 1.540 | 2.168 | 2.672 | 1.928 | 2.805 | 2.538 | 2.596 | 2.811 | 2.603 | 2.939 | 2.208 | 2.339 |
| 004 | 0.213 | 1.000 | 3.402 | 2.445 | 2.395 | 2.385 | 2.555 | 2.282 | 2.471 | 2.130 | 1.788 | 1.928 | 2.530 |
| 005 | 0.188 | 2.393 | 2.618 | 1.996 | 1.781 | 2.346 | 2.172 | 1.962 | 2.430 | 2.470 | 2.924 | 2.805 | 3.410 |
| 006 | 0.000 | 0.576 | 0.643 | 0.821 | 0.611 | 0.583 | 0.574 | 0.440 | 0.809 | 1.058 | 0.960 | 0.875 | 1.327 |
| 007 | 0.489 | 3.610 | 3.363 | 4.480 | 2.729 | 4.306 | 5.240 | 4.918 | 5.453 | 5.125 | 3.887 | 4.413 | 6.614 |
| 008 | 0.335 | 1.200 | 1.913 | 2.822 | 2.951 | 2.702 | 2.664 | 2.440 | 3.458 | 4.268 | 4.571 | 4.285 | 3.762 |
| N | 8 | 8 | 8 | 8 | 8 | 8 | 8 | 8 | 8 | 8 | 8 | 8 | 8 |
| Mean | 0.249 | 1.724 | 2.259 | 2.551 | 1.992 | 2.485 | 2.596 | 2.398 | 2.661 | 2.661 | 2.623 | 2.521 | 3.116 |
| SD | 0.156 | 0.944 | 0.892 | 1.042 | 0.924 | 1.105 | 1.319 | 1.269 | 1.405 | 1.384 | 1.200 | 1.248 | 1.617 |
| SE | 0.055 | 0.334 | 0.315 | 0.368 | 0.327 | 0.391 | 0.466 | 0.449 | 0.497 | 0.489 | 0.424 | 0.441 | 0.572 |
| CV | 62.7 | 54.8 | 39.5 | 40.8 | 46.4 | 44.5 | 50.8 | 52.9 | 52.8 | 52.0 | 45.7 | 49.5 | 51.9 |
| Min | 0.000 | 0.576 | 0.643 | 0.821 | 0.611 | 0.583 | 0.574 | 0.440 | 0.809 | 1.058 | 0.960 | 0.875 | 1.327 |
| Q1 | 0.147 | 1.100 | 1.903 | 2.048 | 1.240 | 1.942 | 2.008 | 1.800 | 1.869 | 1.742 | 1.740 | 1.827 | 2.135 |
| Med | 0.249 | 1.536 | 2.120 | 2.559 | 2.162 | 2.544 | 2.547 | 2.361 | 2.510 | 2.375 | 2.574 | 2.068 | 2.774 |
| Q3 | 0.355 | 2.169 | 2.991 | 2.947 | 2.787 | 3.010 | 2.924 | 2.752 | 3.135 | 3.436 | 3.413 | 3.545 | 3.586 |
| Max | 0.489 | 3.610 | 3.402 | 4.480 | 2.951 | 4.306 | 5.240 | 4.918 | 5.453 | 5.125 | 4.571 | 4.413 | 6.614 |
| GeoM | 0.257 | 1.512 | 2.051 | 2.331 | 1.730 | 2.189 | 2.252 | 2.033 | 2.326 | 2.365 | 2.367 | 2.250 | 2.809 |
| G_CV | 54.7 | 60.6 | 56.2 | 52.2 | 69.6 | 67.0 | 70.2 | 79.2 | 63.6 | 56.3 | 53.8 | 56.3 | 50.8 |
| N\|x > 0 | 7 | 8 | 8 | 8 | 8 | 8 | 8 | 8 | 8 | 8 | 8 | 8 | 8 |

TABLE 10
Oxybutynin:N-desethyloxybutynin - Ratios by Sampling Times

| | Oxybutynin/N-Desethyloxybutynin ratio (Treatment A) | | | | | | | | | |
|---|---|---|---|---|---|---|---|---|---|---|
| | Scheduled time | | | | | | | | | |
| subject | 0 | 24 | 48 | 72 | 96 | 120 | 144 | 168 | | |
| 1 | #DIV/0! | 1.234578 | 1.275218 | 1.114835 | 1.016807 | 0.898612 | 1.369177 | 1.047978 | | |
| 2 | #DIV/0! | 1.335111 | 1.710469 | 1.85077 | 2.406964 | 1.98087 | 1.857727 | 2.532056 | | |
| 3 | #DIV/0! | 0.834332 | 0.779528 | 0.855998 | 1.02758 | 0.985777 | 1.295227 | 1.000655 | | |
| 4 | #DIV/0! | 0.94 | 0.911234 | 1.177933 | 0.879935 | 0.773569 | 2.549745 | 1.894218 | | |
| 5 | #DIV/0! | 0.532629 | 0.540671 | 0.571733 | 0.523357 | 0.542866 | 0.764978 | 0.570886 | | |
| 6 | #DIV/0! | 0.675397 | 1.163725 | 0.817483 | 0.896835 | 0.866186 | 2.962716 | 3.606876 | | |
| 7 | #DIV/0! | 0.715408 | 0.855317 | 0.780554 | 0.730863 | 0.771106 | 0.844011 | 0.63034 | | |
| 8 | #DIV/0! | 0.556104 | 0.574737 | 0.505638 | 0.465163 | 0.431636 | 0.673465 | 0.632159 | | |
| mean | #DIV/0! | 0.85 | 0.98 | 0.96 | 0.99 | 0.91 | 1.54 | 1.49 | Mean | 1.10 |
| SD | #DIV/0! | 0.30 | 0.39 | 0.43 | 0.61 | 0.47 | 0.85 | 1.10 | SD | 0.67 |
| RSD | #DIV/0! | 35.1 | 40.1 | 44.7 | 61.2 | 52.0 | 55.3 | 73.9 | RSD | 60.7 |

| Oxy- | Oxybutynin/N-Desethyloxybutynin ratio (Treatment B) | | | | | | | | | |
|---|---|---|---|---|---|---|---|---|---|---|
| butynin | Scheduled time | | | | | | | | | |
| subject | 0 | 24 | 48 | 72 | 96 | 120 | 144 | 168 | | |
| 1 | 1.09507 | 1.217078 | 1.654863 | 1.010005 | 1.882521 | 1.343303 | 1.65491 | 1.749741 | | |
| 2 | 1.048 | 2.854439 | 1.698213 | 1.61849 | 2.535162 | 1.758631 | 2.465766 | 2.223732 | | |
| 3 | 1.180952 | 0.977922 | 1.002768 | 0.788174 | 0.854772 | 0.827094 | 1.117021 | 0.834972 | | |
| 4 | 0.784038 | 1.33 | 0.886831 | 1.053988 | 0.924426 | 0.960587 | 0.962818 | 0.843874 | | |
| 5 | 0.638298 | 0.656916 | 0.533995 | 0.817635 | 0.975856 | 0.818414 | 1.06814 | 0.829032 | | |
| 6 | #DIV/0! | 0.944444 | 1.121306 | 0.846529 | 1.022913 | 0.782161 | 2.655052 | 1.051997 | | |
| 7 | 0.646217 | 0.708864 | 0.80226 | 0.722768 | 0.685599 | 0.999536 | 1.876145 | 0.802842 | | |
| 8 | 0.465672 | 0.534167 | 0.51333 | 0.553863 | 0.553033 | 0.457809 | 0.813438 | 0.603137 | | |
| mean | #DIV/0! | 1.15 | 1.03 | 0.93 | 1.18 | 0.99 | 1.58 | 1.12 | Mean | 1.14 |
| SD | #DIV/0! | 0.74 | 0.45 | 0.32 | 0.68 | 0.40 | 0.70 | 0.56 | SD | 0.57 |
| RSD | #DIV/0! | 64.2 | 44.0 | 34.6 | 57.4 | 39.9 | 44.7 | 50.5 | RSD | 50.5 |

TABLE 11
Oxybutynin - Treatment A:Treatment B Ratios by Sampling Times
Treatment A/Treatment B Ratio - Oxybutynin

| | Scheduled time | | | | | | | | | |
|---|---|---|---|---|---|---|---|---|---|---|
| subject | 0 | 24 | 48 | 72 | 96 | 120 | 144 | 168 | | |
| 1 | 0 | 1.92857 | 1.02555 | 1.80425 | 2.94673 | 1.94337 | 1.40689 | 1.4424 | | |
| 2 | 0 | 0.34347 | 0.89195 | 0.91794 | 0.89168 | 0.93403 | 0.98459 | 1.59532 | | |
| 3 | 0 | 1.57171 | 1.22953 | 1.31814 | 1.4017 | 1.25474 | 1.55062 | 1.56477 | | |
| 4 | 0 | 1.13083 | 0.6533 | 1.76484 | 1.22809 | 1.18551 | 3.05244 | 2.60843 | | |
| 5 | 0 | 1.95738 | 2.31545 | 3.12561 | 2.3786 | 2.85938 | 4.63405 | 2.07252 | | |
| 6 | #DIV/0! | 1.56434 | 1.64632 | 1.68201 | 2.2672 | 3.69079 | 2.91995 | 6.91404 | | |
| 7 | 0 | 0.99609 | 3.34804 | 2.56609 | 3.21486 | 1.80809 | 0.60706 | 1.95631 | | |
| 8 | 0 | 2.11076 | 2.22403 | 1.63532 | 1.27635 | 2.17947 | 1.46285 | 2.07228 | | |
| mean | #DIV/0! | 1.45 | 1.67 | 1.85 | 1.95 | 1.98 | 2.08 | 2.53 | Mean | 1.93 |
| SD | #DIV/0! | 0.60 | 0.91 | 0.69 | 0.87 | 0.93 | 1.34 | 1.81 | SD | 1.08 |
| RSD | #DIV/0! | 41.1 | 54.6 | 37.5 | 44.5 | 46.8 | 64.7 | 71.6 | RSD | 55.9 |

TABLE 12
N-desethyloxybutynin - Treatment A:Treatment B Ratios by Sampling Times

| | Scheduled time | | | | | | | |
|---|---|---|---|---|---|---|---|---|
| subject | 192 | 216 | 240 | 264 | 288 | 312 | 336 | 360 |

Treatment A/Treatment B Ratio - N-Desethyloxybutynin

| | | | | | | | | | | |
|---|---|---|---|---|---|---|---|---|---|---|
| 1 | 0 | 1.9012 | 1.3309 | 1.6346 | 5.45559 | 2.90507 | 1.70049 | 2.40829 | | |
| 2 | 0 | 0.7343 | 0.8856 | 0.8027 | 0.93917 | 0.82924 | 1.30685 | 1.40106 | | |
| 3 | 0 | 1.8422 | 1.5816 | 1.2137 | 1.16598 | 1.05276 | 1.33727 | 1.30569 | | |
| 4 | 0 | 1.6 | 0.6358 | 1.5791 | 1.29019 | 1.47212 | 1.15264 | 1.16206 | | |
| 5 | 0 | 2.4141 | 2.2869 | 4.4699 | 4.43515 | 4.31074 | 6.47053 | 3.00968 | | |
| 6 | ##### | 2.1875 | 1.5863 | 1.7418 | 2.58592 | 3.33276 | 2.61672 | 2.01658 | | |
| 7 | 0 | 0.987 | 3.1404 | 2.3761 | 3.01576 | 2.34371 | 1.34943 | 2.49168 | | |
| 8 | 0 | 2.0275 | 1.9864 | 1.7913 | 1.51745 | 2.31162 | 1.76689 | 1.97714 | | |
| mean | ##### | 1.71 | 1.68 | 1.95 | 2.55 | 2.32 | 2.21 | 1.97 | Mean | 2.06 |
| SD | ##### | 0.58 | 0.80 | 1.11 | 1.66 | 1.19 | 1.78 | 0.65 | SD | 1.17 |
| RSD | ##### | 34.0 | 47.5 | 57.1 | 65.2 | 51.2 | 80.5 | 33.0 | RSD | 56.7 |

Treatment A/Treatment B Ratio - N-Desethyloxybutynin

| | | | | | | | | | | |
|---|---|---|---|---|---|---|---|---|---|---|
| 1 | 0 | 1.9012 | 1.3309 | 1.6346 | 5.45559 | 2.90507 | 1.70049 | 2.40829 | | |
| 2 | 0 | 0.7343 | 0.8856 | 0.8027 | 0.93917 | 0.82924 | 1.30685 | 1.40106 | | |
| 3 | 0 | 1.8422 | 1.5816 | 1.2137 | 1.16598 | 1.05276 | 1.33727 | 1.30569 | | |
| 4 | 0 | 1.6 | 0.6358 | 1.5791 | 1.29019 | 1.47212 | 1.15264 | 1.16206 | | |
| 5 | 0 | 2.4141 | 2.2869 | 4.4699 | 4.43515 | 4.31074 | 6.47053 | 3.00968 | | |
| 6 | ##### | 2.1875 | 1.5863 | 1.7418 | 2.58592 | 3.33276 | 2.61672 | 2.01658 | | |
| 7 | 0 | 0.987 | 3.1404 | 2.3761 | 3.01576 | 2.34371 | 1.34943 | 2.49168 | | |
| 8 | 0 | 2.0275 | 1.9864 | 1.7913 | 1.51745 | 2.31162 | 1.76689 | 1.97714 | | |
| mean | ##### | 1.71 | 1.68 | 1.95 | 2.55 | 2.32 | 2.21 | 1.97 | Mean | 2.06 |
| SD | ##### | 0.58 | 0.80 | 1.11 | 1.66 | 1.19 | 1.78 | 0.65 | SD | 1.17 |
| RSD | ##### | 34.0 | 47.5 | 57.1 | 65.2 | 51.2 | 80.5 | 33.0 | RSD | 56.7 |

TABLE 13
Multiple-dose pharmacokinetic parameters after 20 days (steady-state)
topical administration of 42 mg (A), 60 mg (B), or 84 mg (C)
oxybutynin (OXY) and N-desethyloxybutynin (DEO) in transdermal
gel containing 3% (w/w) of the active substance
(mean ± SD, N = 16 for each arm).

| | OXY | DEO |
|---|---|---|
| Treatment A: 42 mg oxybutynin | | |
| Cmax (ng/mL) | 4.4 ± 1.9 | 4.6 ± 3.9 |
| Cav (ng/mL) | 3.0 ± 1.0 | 3.0 ± 1.3 |
| tmax (h) | 11.8 ± 10.0 | 8.3 ± 7.4 |
| AUC0-24 (ng · h/mL) | 72.5 ± 24.3 | 73.1 ± 31.8 |
| Ratio DEO:OXY (Cav) | 1.0 | |
| Treatment B: 60 mg oxybutynin | | |
| Cmax (ng/mL) | 6.3 ± 3.7 | 4.9 ± 2.6 |
| Cav (ng/mL) | 4.3 ± 2.4 | 4.0 ± 2.1 |
| tmax (h) | 15.8 ± 7.9 | 7.9 ± 7.0 |
| AUC0-24 (ng · h/mL) | 102.8 ± 56.7 | 95.9 ± 51.6 |
| Ratio DEO:OXY (Cav) | 0.9 | |
| Treatment C: 84 mg oxybutynin | | |
| Cmax (ng/mL) | 7.3 ± 2.3 | 8.6 ± 4.9 |
| Cav (ng/mL) | 5.4 ± 1.6 | 7.0 ± 4.0 |
| tmax (h) | 12.4 ± 8.3 | 12.8 ± 9.2 |
| AUC0-24 (ng · h/mL) | 130.1 ± 38.3 | 167.0 ± 96.9 |

| | |
|---|---|
| Ratio DEO:OXY (Cav) | 1.3 |

TABLE 14
Single-dose pharmacokinetic parameters after one day topical
administration of 84 mg oxybutynin. Data is dose-normalized and
represent arithmetic mean, standard deviation (±SD), or
median with range (for Tmax only) of 25 subjects for each skin site

| | OXY | DEO |
|---|---|---|
| Site A: Abdomen | | |
| AUC0-120 h (ng · h/mL) | 284 ± 108 | 636 ± 289 |
| AUC0-∞ (ng · h/mL) | 298 ± 108 | 664 ± 293 |
| Cmax (ng/mL) | 6.3 ± 3.5 | 14.5 ± 7.4 |
| Tmax (h) median | 24 (4-48) | 24 (8-37) |
| Site B: Thighs | | |
| AUC0-120 h (ng · h/mL) | 287 ± 145 | 645 ± 371 |
| AUC0-∞ (ng · h/mL) | 310 ± 152 | 688 ± 386 |
| Cmax (ng/mL) | 5.8 ± 2.6 | 12.8 ± 7.7 |
| Tmax (h) median | 36 (12-48) | 36 (12-72) |
| Site C: Shoulders | | |
| AUC0-120 h (ng · h/mL) | 329 ± 139 | 607 ± 263 |
| AUC0-∞ (ng · h/mL) | 342 ± 139 | 634 ± 275 |
| Cmax (ng/mL) | 8.8 ± 5.5 | 12.9 ± 5.5 |
| Tmax (h) median | 24 (8-48) | 24 (12-48) |

TABLE 15
Cₘₐₓ and AUC₀₋₁₂₀ of both R- and S-enantiomers of OXY and
DEO after single-dose topical administration of 84 mg OXY in
the investigational gel formulation to the abdominal area. Data was
normalized to the nominal 84 mg dose (mean ± SD, N = 10).

| | Pharma- | | | |
|---|---|---|---|---|
| | cokinetic | | | |
| Analyte | Parameter | R | S | R/S |
| OXY | Cₘₐₓ (ng/mL) | 3.6 ± 1.6 | 5.8 ± 2.6 | 0.6 ± 0.04 |
| | AUC₀₋₁₂₀ | 151.1 ± 56.0 | 246.0 ± 96.7 | 0.6 ± 0.04 |
| | (ng · h/mL) | | | |
| DEX | Cₘₐₓ (ng/mL) | 6.3 ± 3.5 | 6.9 ± 3.8 | 0.9 ± 0.3 |
| | AUC₀₋₁₂₀ | 269.5 ± 132.8 | 285.5 ± 152.9 | 1.0 ± 0.3 |
| | (ng · h/mL) | | | |

While the invention has been described and pointed out in detail with reference to operative embodiments thereof, it will be understood by those skilled in the art that various changes, modifications, substitutions, and omissions can be made without departing from the spirit of the invention. It is intended therefore, that the invention embrace those equivalents within the scope of the claims that follow.

What is claimed is:

1. A composition for administration of anti-cholinergic agent comprising:
an anti-cholinergic agent in an amount between about 1 to 5% by weight of the composition;
a urea-containing compound of urea, 1,3-dimethylurea, 1,1-diethylurea, 1-acetyl-1-phenylurea, isopropylideneurea, allophanic acid, hydantoic acid, allophanoyl, pyrrolidone carboxylic acid, biuret, thiobiuret, dithiobiuret, triuret or 2-(3-methylureido)-1-naphthoic acid in an amount between about 1 to 10% by weight of the composition; and
a carrier present in an amount between about 40 to 80 percent by weight of the composition, wherein the carrier comprises the combination of an alcohol, a polyalcohol, water, and a monoalkyl ether of a diethylene glycol or a tetraglycol furol,
wherein the alcohol is present in an amount of about 30 to 70% by weight of the composition, the polyalcohol is present in an amount of about 10 to 20% by weight of the composition, and the monoalkyl ether of diethylene glycol or tetraglycol furol is present in an amount of about 1 to 10% by weight of the composition.

2. The composition of claim 1, wherein the anticholinergic agent is at least one of oxybutynin, flavoxate, imipramine, propantheline, phenylpropanolamine, darifenacin, duloxetine, tolterodine tartrate, trospium, or solifenacin succinate or a pharmaceutically acceptable salt thereof.

3. The composition of claim 1, wherein the anticholinergic agent is oxybutynin which is present as oxybutynin, or oxybutynin free base, as a racemate, an isomer, or as a pharmaceutically acceptable salt thereof.

4. The composition of claim 1, wherein the pharmaceutically acceptable salt of oxybutynin is selected from the group consisting of acetate, bitartrate, citrate, edetate, edisylate, estolate, esylate, fumarate, gluceptate, gluconate, glutamate, hydrobromide, hydrochloride, lactate, malate, maleate, mandelate, mesylate, methylnitrate, mucate, napsylate, nitrate, pamoate, pantothenate, phosphate, salicylate, stearate, succinate, sulfate, tannate and tartrate.

5. The composition of claim 1, further comprising at least one excipient selected from the group consisting of gelling agents, solvents, antimicrobials, preservatives, antioxidants, buffers, humectants, sequestering agents, moisturizers, emollients, or permeation enhancers.

6. The composition of claim 1, in the form of a gel, ointment, cream, foam, lotion, liposome, micelle, microsphere, lacquer, patch, bandage, dressing, spray, aerosol, solution, emulsion, nanosphere, microcapsule, nanocapsule or a combination thereof.

7. The composition of claim 1, in a form of a gel, wherein the urea-containing compound is urea; the alcohol is ethanol, propanol, isopropanol, 1-butanol, 2-butanol or a mixture thereof; the polyalcohol is propylene glycol, dipropylene glycol or a mixture of thereof; the monoalkyl ether of diethylene glycol, when present, is diethylene glycol monomethyl ether, diethylene glycol monoethyl ether or a mixture thereof, and the tetraglycol, when present, is glycofurol.

8. The composition of claim 7, wherein the polyalcohol is present in an amount of no more than about 15% by weight of the composition, and the monoalkyl ether of diethylene glycol or tetraglycol furol, whichever is included, is present in an amount of about 1 to 5% by weight of the composition.

9. A method for treating overactive bladder or urge and urinary incontinence in a subject, the method comprising topically or transdermally administering to the skin or the mucosa of a subject in need thereof, a composition according to claim 1.

10. The method of claim 9, wherein the anticholinergic agent is oxybutynin which is present as oxybutynin or oxybutynin free base as a racemate, isomer, or pharmaceutically acceptable salt thereof.

11. The method of claim 9, wherein the administration to a subject of a daily dose of about 30 mg to 90 mg of oxybutynin delivers a mean cumulative plasmatic daily dose of oxybutynin ranging from about 0.5 to about 10 mg over a period of 24 hours.

12. The method of claim 9, wherein the administration to a subject of a daily dose of about 30 mg to 90 mg of oxybutynin provides a mean plasma area under the curve (AUC) of oxybutynin ranging from about 40 to about 500 ng*h/mL over a period of 24 hours.

13. The method of claim 9, wherein the ratio between peak plasma concentration of oxybutynin (Cmax) and steady state plasmatic level of oxybutynin (Cavg) is 1.3 to 1.5.

14. The method of claim 9, wherein the transdermal composition is administered for a duration of from about 24 to about 72 hours.

15. A method for treating overactive bladder or urge and urinary incontinence in a subject, the method comprising administering to a subject in need thereof, a composition consisting essentially of:
oxybutynin, oxybutynin free base or a pharmaceutically acceptable salt of oxybutynin present in an amount between about 1 to 5% by weight of the composition;
urea present in an amount of about 1 to 10% by weight of the composition; and
a carrier present in an amount of about 40 to 80 percent by weight of the composition, wherein the carrier consists essentially of the combination of:
an alcohol selected from the group consisting of ethanol, propanol, isopropanol, 1-butanol, 2-butanol or a mixture thereof,
a polyalcohol selected from the group consisting of propylene glycol, dipropylene glycol or a mixture thereof,
a monoalkyl ether of diethylene glycol selected from the group consisting of diethylene glycol monomethyl ether, diethylene glycol monoethyl ether or a mixture thereof, or a tetraglycol furol, and
water,
wherein the alcohol is present in an amount of about 30 to 70 percent by weight of the composition, the polyalcohol is present in an amount of about 10 to 20% by weight of the composition, and the monoalkyl ether of diethylene glycol or a tetraglycol furol, whichever is included, is present in an amount of about 1 to 10% by weight of the composition.

16. The method of claim 15, wherein the composition contains a gelling agent and is in the form of a gel, the urea-containing compound is urea; the alcohol is ethanol, propanol, isopropanol, 1-butanol, 2-butanol or a mixture thereof; the polyalcohol is propylene glycol, dipropylene glycol or a mixture of thereof; the monoalkyl ether of diethylene glycol, when present, is diethylene glycol monomethyl ether, diethylene glycol monoethyl ether or a mixture thereof, and the tetraglycol, when present, is glycofurol.

17. The method of claim 15, wherein the composition is administered upon the abdomen, shoulder, or thigh of the subject.

18. The method of claim 15, wherein the applied daily dose of oxybutynin is between 0.06 and 0.18 mcg per cm².

19. The method of claim 15, wherein the polyalcohol is present in an amount of no more than about 15% by weight of the composition, and the monoalkyl ether of diethylene glycol or tetraglycol furol, whichever is included, is present in an amount of about 1 to 5% by weight of the composition.
